SEQUENCE LISTING (8 sequences)

SEQ ID NO: 1 - DNA, 24 bp, synthetic construct
  source 1..24: mol_type = other DNA
tgccactggtgagaaacctgagat

SEQ ID NO: 2 - DNA, 24 bp, synthetic construct
  source 1..24: mol_type = other DNA
tcagagacaaaggctgagcaggtt

SEQ ID NO: 3 - DNA, 20 bp, synthetic construct
  source 1..20: mol_type = other DNA
ggggcttatgtcttgccagt

SEQ ID NO: 4 - DNA, 24 bp, synthetic construct
  source 1..24: mol_type = other DNA
agaggtttccattggttacttggt

SEQ ID NO: 5 - DNA, 20 bp, synthetic construct
  source 1..20: mol_type = other DNA
aggatgtgggcatctttggg

SEQ ID NO: 6 - DNA, 20 bp, synthetic construct
  source 1..20: mol_type = other DNA
gagatagcagccaggcacaa

SEQ ID NO: 7 - DNA, 20 bp, synthetic construct
  source 1..20: mol_type = other DNA
tgagactagcctggacacca

SEQ ID NO: 8 - DNA, 20 bp, synthetic construct
  source 1..20: mol_type = other DNA
aagctcactgcagcctcaaa

(19) United States
(12) Patent Application Publication
(10) Pub. No.: US 2026/0248836 A1
(43) Pub. Date: Aug. 27, 2026

(54) COMBINATION THERAPY OF miR-99b-5p AND ANDROGEN RECEPTOR ANTAGONISTS FOR TREATING CASTRATION-RESISTANT PROSTATE CANCER

(71) Applicant: UNIVERSITY OF MARYLAND, EASTERN SHORE, Princess Anne, MD (US)

(72) Inventors: BI-DAR WANG, Salisbury, MD (US); MOHAMMAD WASEEM, Princess Anne, MD (US)

(21) Appl. No.: 19/373,974

(22) Filed: Oct. 30, 2025

Related U.S. Application Data
(60) Provisional application No. 63/717,091, filed on Nov. 6, 2024.
(60) Provisional application No. 63/787,429, filed on Apr. 11, 2025.

(51) Int. Cl. A61K 31/7088; A61K 31/4166; A61K 31/58; A61P 35/00; C12N 15/113; C12Q 1/6886

(52) U.S. Cl. CPC A61K 31/7088; A61K 31/4166; A61K 31/58; A61P 35/00; C12N 15/1137; C12Q 1/6886; C12N 2310/141; C12Q 2600/178

(57) ABSTRACT

Downregulated miR-99b-5p and upregulated mTOR cooperatively promotes the African American (AA) PCa aggressiveness and drug resistance. Nuclear mTOR, AR, and SMARCD1 are highly expressed in AA PCa (MDA PCa 2b) compared to EA PCa (LNCaP) cell line. miR-99b-5p inhibited protein levels of mTOR, AR/AR-V7 and SMARCD1 in cytoplasm and nuclei of EA and AA PCa. miR-99b-5p effectively inhibits cell proliferation/survival and induced cell apoptosis in EA and AA PCa cells. Moreover, combination of miR-99b-5p and enzalutamide (Enz) synergistically enhances the cytotoxicity against aggressive AA PCa and castration resistant prostate cancer (CRPC). miR-99b-5p or miR-99b-5p/Enz significantly reduces the recruitment of mTOR to the genes involved in the metabolic reprogramming in CRPC. miR-99b-5p can function as an epigenomic driver to modulate the mTOR/AR/SMARCD1 signaling axis in AA PCa and resistant CRPC. miR-99b-5p can be utilized as a biomarker for identifying the presence of prostate cancer.

### CROSS REFERENCE TO RELATED APPLICATIONS

This application claims priority under 35 U.S.C. § 119(e) to provisional patent applications U.S. Ser. No. 63/717,091, filed Nov. 6, 2024, and U.S. Ser. No. 63/787,429, filed Apr. 11, 2025. The provisional patent applications are hereby incorporated by reference in its entirety herein, including without limitation: the specification, claims, and abstract, as well as any figures, tables, appendices, or drawings thereof.

### STATEMENT REGARDING FEDERALLY SPONSORED RESEARCH OR DEVELOPMENT

This invention was made with government support under 5SC1GM127256 awarded by the National Institutes of Health. The government has certain rights in the invention.

### REFERENCE TO SEQUENCE LISTING SUBMITTED ELECTRONICALLY

The instant application contains a Sequence Listing which has been submitted electronically in XML format and is herein incorporated by reference in its entirety. Said XML copy, created on Mar. 2, 2026, is named “P15025US02_SequenceListing.xml” and is 8,262 bytes in size.

### TECHNICAL FIELD

The present disclosure relates generally to functional involvements of the epigenomic driver, miR-99b-5p, in cancer disparities across different demographics and the metabolic reprogramming in cancer. More particularly, but not exclusively, the present disclosure relates to a combination therapy of miR-99b-5p and androgen receptor antagonists for treating castration-resistant prostate cancer.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Prostate cancer (PCa) has been considered as one of the leading cancer-associated mortalities among men in the globe. In the United States, PCa is the most frequently diagnosed cancer (299,010 estimated new cases in 2024) and the second leading cause of cancer deaths (35,250 estimated deaths in 2024) among men. Notably, African American (AA) men exhibit 1.7-time higher PCa incidence and 2.3-time higher PCa mortality rates, when compared to European American (EA) men. Besides the socioeconomic factors, multiple genetic/epigenetic factors have been associated with the PCa disparities observed between EA and AA PCa.

Emergent evidence suggests that alteration in epigenetic/epigenomic mechanisms may play critical roles in promoting the AA PCa disparities. For instance, differential DNA methylation patterns, and deregulated microRNA (miRNA) regulatory networks have been implicated in promoting the AA PCa disparities. See e.g., Devaney et al., “Genome-wide differentially methylated genes in prostate cancer tissues from African American and Caucasian men” Epigenetics (2015) 10(4):319-28; Gujrati et al., “MicroRNA-mRNA regulatory network mediates activation of mTOR and VEGF signaling in African American prostate cancer” Int J Mol Sci (2022) 23(6):2926; and Wang et al., “Identification and functional validation of reciprocal microRNA-mRNA pairings in African American prostate cancer disparities” Clin Cancer Res (2015) 21(21):4970-84; each of which are hereby incorporated by reference in their entireties herein.

MiRNA are short non-coding RNAs regulating protein expression through degradation of target mRNAs or inhibition of protein translation. Emerging evidence has implicated miRNAs as potential biomarkers in cancers, including PCa. A correlation-based approach, combining miRNA and mRNA (or protein) profiling data with mRNA target prediction to identify the miRNAs and mRNA targets with inverse regulatory correlation, has proved to be a more effective way to identify critical miRNA-mRNA interactions in cancers. The present inventors have previously identified dozens of the ‘reciprocal miRNA-mRNA pairings’ that are differentially expressed between AA PCa and EA PCa. See Wang et al., supra. These reciprocal miRNA-mRNA pairings demonstrated significant regulatory effects on activating multiple oncogenic pathways such as ERBB, mTOR, VEGF and HIF-1α signaling, in AA PCa. See Wang et al., supra, and Gujrati et al. “Downregulation of miR-99b-5p and upregulation of nuclear mTOR cooperatively promotes the tumor aggressiveness and drug resistance in African American prostate cancer”, Int J Mol Sci (2022) 23(17):9643, which is hereby incorporated by reference in its entirety herein.

Among these miRNA-mRNA pairing, tumor suppressive miR-99b-5p negatively regulates MTOR expression (at mRNA and protein levels) and play a central role in regulating PI3K/AKT/mTOR/HIF-la/VEGF signaling axis. See Gujrati et al., “MicroRNA-mRNA . . . ”, supra. miR-99b-5p also targets/inhibits AR (encoding androgen receptor, AR) expression at protein level, consequently inhibiting AR/mTOR signaling and AR/mTOR translocation to the nucleus. See Gujrati et al., “Downregulation . . . ”, supra. Since nuclear AR and mTOR transcriptionally activate hundreds of genes in AA PCa and CRPC, miR-99b-5p potentially functions as an epigenomic driver mediating the AA PCa aggressiveness and drug resistance.

In PCa pathogenesis, androgen-receptor (AR) has been shown as a key player for early detection of the disease. AR signaling axis play a critical functional role for normal male reproductive function, and is regulated in subsequent binding of androgens (such as dihydrotestosterone, DHT) to its specific receptor, causing its nuclear events followed by the regulation of transcriptional targets such as prostate-specific antigen (PSA) and transmembrane protease, serine 2 (TMPRSS2). Androgen deprivation therapy (ADT) has been a systemic treatment of both localized and advanced PCa. Unfortunately, a considerable majority of ADT targeted patients ultimately progress to castration-resistant prostate cancer (CRPC) in a therapeutic window of 2-3 years. CRPC has also been characterized by constant tumor growth despite the castrated levels of serum testosterone, ultimately leading to significant patient deaths. Several AR antagonists, such as enzalutamide (Enz, a nonsteroidal antiandrogen), and abiraterone acetate (an irreversible inhibitor of CYP17A1) were approved by FDA for therapeutic alternative for aggressive and metastatic PCa. However, a group of patients have shown a resistance to both Enz and abiraterone due to the expression of oncogenic AR splice variants, such as AR-V7. AR is a 110-kDa transcription factor classified in the superfamily of steroid hormone receptor. The phosphoinositide 3-kinase (PI3K)-AKT-mTOR signaling pathway has clearly demonstrated as a core mechanism that regulates ADT resistance and triggers tumor growth at castrated levels of testosterone. Indeed, this regulatory pathway has been shown as alterations at both genomic and transcriptional level in almost all aggressive PCa (26). Therefore, cancer cells are actively utilizing this pathway to adapt to the cellular stress triggered by ADT.

Furthermore, recent investigations have established a direct link between mTOR and AR signaling, conveying a progressive reciprocation on these molecular targets during the development of androgen insensitivity. AR and mTOR have been confirmed as direct targets of miR-99b-5p. Overexpression of miR-99b-5p inhibits AR, mTOR, and PSA expression levels, leading to inhibition of cell proliferation/migration, induction of autophagy and apoptosis. Additionally, it has been proposed that AR and mTOR co-activate a set of downstream genes involved in the metabolic reprogramming critical for CRPC progression. SMARCD1, a member of the SWI/SNF family proteins, is known as a cofactor of AR involved in activation of AR-target genes.

Interestingly, miR-99b-5p also targets and negatively regulates SMARCD1 expression. Recent studies have further demonstrated that SMARCD1 can regulate AR-target genes in androgen-dependent and -independent manners, suggesting its potential involvement in progression to CRPC. Taken together, miR-99b-5p (downregulated in AA PCa and CRPC) may play a pivotal role in regulating mTOR/AR/SMARCD1 signaling axis in aggressive PCa.

Thus, there exists a need in the art to study the functional involvements of the epigenomic driver, miR-99b-5p, in AA PCa disparities and the metabolic reprogramming in CRPC.

### SUMMARY

Prostate cancer (PCa) is the most frequently diagnosed cancer and second leading cause of cancer deaths among American men. African American (AA) men exhibited 1.7-fold higher PCa incidence and 2.3-fold higher PCa mortality rates when compared to the European American (EA) men. Besides the socioeconomic factors, emerging evidence has highlighted that biological risk factors may play critical roles in the AA PCa disparities. Previously, the present inventors showed that downregulated miR-99b-5p and upregulated mTOR cooperatively promotes the AA PCa aggressiveness and drug resistance.

In the present disclosure, the functional involvements of the epigenomic driver, miR-99b-5p, were studied in AA PCa disparities and the metabolic reprogramming in CRPC. A series of in vitro biochemistry, cellular and molecular biology, and functional assays were performed to elucidate the molecular mechanism of miR-99b/mTOR/AR/SMARCD1 signaling in AA PCa and CRPC.

First, immunofluorescence assays were employed to explore the expression levels and subcellular distributions of mTOR, AR, ARV7, and SMARCD1 in 2D (monolayer) and 3D (organoid) in PCa cell line models derived from EA PCa (LNCaP, PC-3, DU-145, and CRPC lines 22Rv1 and C4-2B) and AA PCa (MDA PCa 2b).

Second, the EA and AA PCa cell lines were transfected with nonsense control RNA, miR-99b-5p mimic, Enz, or miR-99b-5p/Enz combination, then followed by immunofluorescence assays and western blot analysis. Specifically, immunofluorescence assays were used to visualize the protein expression and distribution under the four treatments, while the western blot assays were used to examine the protein levels of mTOR, AR, pAR and SMARCD1 in cytoplasmic and nuclear fractions of the EA and AA PCa cells under these treatments.

Third, functional assays (MTT, colony forming/clonogenic, and TUNEL assays) were conducted in 2D and 3D cell cultures to examine the functional impacts of miR-99b-5p and Enz as single agents, and miR-99b-5p/Enz as combination therapy. Finally, chromatin immunoprecipitation-qPCR assays were used to investigate the binding affinity of mTOR/AR complex on their downstream target genes under the treatments.

In summary, the present disclosure demonstrates the functional effects of miR-99b-5p and miR-99b-5p/Enz combination in suppressing EA PCa, CRPC and AA PCa. The results show miR-99b-5p-mediated mTOR/AR/SMARCD1 signaling plays a central role in promoting the aggressiveness and drug resistance in CRPC and AA PCa. Therefore, miR-99b-5p serves as a precision prognostic biomarker and therapeutic tool for detecting and targeting the aggressive AA PCa and CRPC.

Androgen deprivation therapy (ADT) has been systemically applied as a first-line therapy for PCa patients. Despite the initial responses, the majority of patients under ADT eventually experienced tumor progression to castration-resistant prostate cancer (CRPC), further leading to tumor metastasis to distant organs. The next generation hormone therapeutic drugs, enzalutamide (Enz) and abiraterone acetate (Abi) have been applied to the metastatic PCa patients, and showed superior efficacies compared to the traditional ADT drugs. However, the resistance (genetic and acquired resistance) to Enz and Abi remains as a major clinical challenge for aggressive/metastatic PCa and CRPC. Therefore, identifying the key molecular mechanisms underlying PCa metastasis remains crucial for the development of novel therapies for metastatic PCa and CRPC.

Previously, the present inventors identified that tumor-suppressive miR-99b-5p is frequently downregulated in aggressive African American (AA) PCa and CRPC, leading to upregulation of mTOR, androgen receptor (AR), and HIF-la signaling. The present disclosure tests the drug efficacies of Enz and Abi as single agents and in combination with tumor suppressive miR-99b-5p mimic. Our results have demonstrated that miR-99b-5p simultaneously targets mTOR and AR signaling, significantly sensitizing the PCa cells to Enz and Abi. Specifically, the combination of Enz/Abi and miR-99b-5p mimic significantly synergize the inhibitory efficacies of the drugs to suppress EMT-mediated metastasis and angiogenesis of aggressive PCa, including CRPC and AA PCa. This novel therapeutic strategy, by combining tumor suppressive miRNA (miR-99b-5p mimic) and new generation hormone therapeutic drugs (Enz and Abi), is scientifically and clinically impactful for the development of new therapies for the aggressive types of PCa (i.e. metastatic PCa and CRPC), and any AR overexpressing cancers in general.

The following objects, features, advantages, aspects, and/or embodiments are not exhaustive and do not limit the overall disclosure. No single embodiment needs to provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of the present disclosure to explore the miR-99b-5p/mTOR/AR/SMARCD1 signaling axis in AA PCa aggressiveness. The analyses used in the present disclosure included immunofluorescence, western blot, in-vitro functional assays (TUNEL, colony forming, and MTT), and chromatin immunoprecipitation (ChIP)-qPCR assays in 2D and/or 3D culture model of EA PCa and AA PCa cell lines.

Specifically, the immunofluorescence staining, and western blot analysis revealed that nuclear mTOR, AR, and SMARCD1 were highly expressed in AA PCa (MDA PCa 2b) compared to EA PCa (LNCaP) cell line. Western blot analysis further revealed that miR-99b-5p inhibited protein levels of mTOR, AR/AR-V7 and SMARCD1 in cytoplasm and nuclei of EA and AA PCa. The in-vitro functional (MTT, TUNEL, and clonogenic) assays have demonstrated that miR-99b-5p effectively inhibited cell proliferation/survival and induced cell apoptosis in EA and AA PCa cells. Moreover, combination of miR-99b-5p and enzalutamide (Enz) synergistically enhances the cytotoxicity against aggressive AA PCa and castration resistant prostate cancer (CRPC). mTOR ChIP-qPCR assays further demonstrated that miR-99b-5p or miR-99b-5p/Enz significantly reduces the recruitment of mTOR to the genes involved in the metabolic reprogramming in CRPC.

It is still yet a further object, feature, and/or advantage of the present disclosure to utilize miR-99b-5p as an epigenomic driver to modulate the mTOR/AR/SMARCD1 signaling axis in AA PCa and resistant CRPC.

miR-99b-5p as disclosed herein can be used in a wide variety of applications.

Methods can be practiced which facilitate use of the tumor suppressive regulator that includes miR-99b-5p, manufacture of therapeutics (e.g., drugs), and maintenance of therapeutics, which accomplish some or all of the previously stated objectives.

The tumor suppressive regulator that includes miR-99b-5p can be incorporated into systems or kits which accomplish some or all of the previously stated objectives.

According to some aspects of the present disclosure, a tumor suppressive regulator comprises miR-99b-5p, wherein the miR-99b-5p functions as an epigenomic regulator.

According to some additional aspects of the present disclosure, the tumor suppressive regulator further comprises enzalutamide (Enz) to enhance cytotoxicity of the tumor suppressive regulator against cancer.

According to some additional aspects of the present disclosure, the miR-99b-5p regulates a cellular process. The cellular process can be selected from a group consisting of: cell growth, cell proliferation, cell motility, cell survival, protein synthesis, and autophagy. The cellular process can also be transcription and the miR-99b-5p regulates the transcription of a gene by altering a chromatin structure of the gene. The gene can be the SMARCD1 gene.

According to some additional aspects of the present disclosure, the miR-99b-5p regulates a nuclear receptor by binding a hormone thereto. The nuclear receptor can be the androgen receptor (AR) and the hormone is selected from the group consisting of testosterone and dihydrotestosterone.

According to some additional aspects of the present disclosure, the miR-99b-5p modulates the mTOR/AR/SMARCD1 signaling axis.

According to some other aspects of the present disclosure, a method of treating cancer comprises inhibiting cell proliferation/survival using miR-99b-5p; and inducing cell apoptosis in cancer cells.

According to some additional aspects of the present disclosure, the method is practiced in vitro or in vivo.

According to some additional aspects of the present disclosure, the cancer is prostate cancer.

According to some other aspects of the present disclosure, a method of diagnosing cancer comprises using miR-99b-5p as a biomarker to identify presence of prostate cancer.

According to some additional aspects of the present disclosure, the method further comprises inhibiting cell proliferation/survival using the miR-99b-5p.

According to some additional aspects of the present disclosure, the method further comprises inducing cell apoptosis in cancer cells.

According to some additional aspects of the present disclosure, the method further comprises determining the cancer is a castration resistant cancer.

According to some additional aspects of the present disclosure, the method further comprises castrating an area of the body infected by the cancer.

According to some additional aspects of the present disclosure, the method further comprises treating the cancer with a hormone-based treatment.

According to some additional aspects of the present disclosure, the method further comprises further comprising enhancing cytotoxicity against the cancer with enzalutamide (Enz).

According to some other aspects of the present disclosure, a therapeutic combination comprises miR-99b-5p and an androgen receptor antagonist that simultaneously targets an androgen receptor (AR) and mammalian target of rapamycin (mTOR) signaling.

According to some additional aspects of the present disclosure, the androgen receptor antagonist comprises enzalutamide (Enz) or abiraterone (Abi).

According to some additional aspects of the present disclosure, the miR-99b-5p and the androgen receptor antagonist inhibit epithelial-mesenchymal transition (EMT) in prostate cancer (PCa); the miR-99b-5p and the androgen receptor antagonist are configured to upregulate E-cadherin; the miR-99b-5p and the androgen receptor antagonist are configured to downregulate Snail; the miR-99b-5p and the androgen receptor antagonist are configured to downregulate N-cadherin; and/or the miR-99b-5p and the androgen receptor antagonist are configured to downregulate Vimentin.

According to some additional aspects of the present disclosure, the miR-99b-5p regulates a cellular process. The cellular process can be selected from a group consisting of: cell growth, cell proliferation, cell motility, cell survival, protein synthesis, and autophagy. The cellular process can be transcription and the miR-99b-5p can regulate the transcription of a gene by altering a chromatin structure of the gene.

According to some additional aspects of the present disclosure, the miR-99b-5p regulates a nuclear receptor by binding a hormone thereto.

According to some additional aspects of the present disclosure, the miR-99b-5p modulates the mTOR/AR/SMARCD1 signaling axis.

According to some other aspects of the present disclosure, a method of treating cancer comprises inhibiting cell proliferation/survival using miR-99b-5p; and simultaneously targeting an androgen receptor (AR) and mammalian target of rapamycin (mTOR) signaling with an androgen receptor antagonist.

According to some additional aspects of the present disclosure, the method is practiced in vitro or in vivo.

According to some additional aspects of the present disclosure, the cancer is prostate cancer.

According to some other aspects of the present disclosure, a method of diagnosing cancer comprises using miR-99b-5p as a biomarker to identify presence of prostate cancer; and determining the cancer is a castration resistant cancer after simultaneously targeting an androgen receptor (AR) and mammalian target of rapamycin (mTOR) signaling with an androgen receptor antagonist.

According to some additional aspects of the present disclosure, the method further comprises inhibiting cell proliferation/survival using the miR-99b-5p.

According to some additional aspects of the present disclosure, the method further comprises inducing cell apoptosis in cancer cells.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIGS. 1A-1C show that immunofluorescence staining revealed the cellular localizations and expression levels of AR, AR-V7, mTOR, and SMARCD1 in EA PCa and AA PCa cell lines.

FIG. 1A shows that immunofluorescence staining showed mTOR (red fluorescence) and AR (green fluorescence) signals in EA PCa cell lines (22Rv1, LNCaP, C4-2B, PC-3 and DU-145), and AA PCa cell lines (MDA PCa 2b). Nuclei were visualized by counterstaining with DAPI (blue fluorescence). Merged images were acquired by imposing DAPI, mTOR and AR signals to depict whether colocalization (yellow) of both mTOR and AR was observed either in nuclei or cytoplasm.

FIG. 1B shows that immunofluorescence demonstrating AR-V7 (green fluorescence) and mTOR (red fluorescence) signals in EA PCa (22Rv1, LNCaP, C4-2B, PC-3 and DU-145) and AA PCa cell lines (MDA PCa 2b). Nuclei were visualized by counterstaining with DAPI (blue fluorescence). Merged images were achieved by overlaying DAPI, mTOR and AR-V7 signals to depict the colocalization (yellow) of both AR-V7 and mTOR either in nuclei or cytoplasm.

FIG. 1C shows immunofluorescence depicting SMARCD1 (green fluorescence) and AR (red fluorescence) signals in EA PCa cell lines (22Rv1, LNCaP, C4-2B, PC-3 and DU-145), and AA PCa cell lines (MDA PCa 2b). Merged images were achieved by overlaying DAPI, SMARCD-1 and AR signals to depict the colocalization (yellow) of both SMARCD-1 and AR either in nuclei or cytoplasm. All the captured fluorescent images were analyzed by using CellScans software V1.18. Five to six random images were captured by using 200× magnification. An artisan of ordinary skill in the art need not view, within isolated figure(s), the near infinite distinct combinations of features described in the following detailed description to facilitate an understanding of the present disclosure.

FIGS. 2A-2B show Western blot analyses of proteins expression levels of AR, AR-V7, mTOR, and SMARCD1 in EA PCa (22Rv1, LNCaP, C4-2B, PC-3 and DU-145) and AA PCa (MDA PCa 2b) cell lines. FIG. 2A shows representative western blot images and FIG. 2B shows quantification of AR, AR-V7, mTOR, and SMARCD1 protein levels from total cell lysates (Total), cytoplasmic lysates (Cytoplasm), and nuclear lysates (Nucleus) of the EA and AA PCa cells were presented. Total lysate, cytoplasmic and nuclear protein fractions were prepared and subjected to the western blot assays (n=3) of the protein indicated above. GAPDH and Lamin B1 were used as endogenous controls for cytoplasmic and nuclear proteins, respectively. The normalized protein level was determined by normalization of the intensity of AR/AR-V7, mTOR or SMARCD1 to the intensity of its corresponding control (GAPDH for Total or Cytoplasm, or Lamin B1 for Nucleus).

FIGS. 3A-3C show bright-field imaging and immunofluorescence staining for 3D cultures derived from EA and AA PCa cell lines.

FIG. 3A shows morphological changes of organoids derived from EA PCa (PC-3, DU-145, LNCaP, 22Rv1, and C4-2B) and AA PCa (MDA PCa 2b), at different time points up to 14 days.

FIG. 3B shows that immunofluorescence showing mTOR (red fluorescence) and AR (green fluorescence) signals in 3D cultures developed from EA PCa (22Rv1, LNCaP, C4-2B, PC-3 and DU-145) and AA PCa (MDA PCa 2b) cell lines. Nuclei were visualized by counterstaining with DAPI (blue fluorescence). Merged images were acquired by imposing DAPI, mTOR and AR signals to evaluate the colocalization (yellow) of mTOR and AR in nuclei or cytoplasm.

FIG. 3C shows that immunofluorescence showing AR (red fluorescence) and SMARCD1 (green fluorescence) signals in organoids developed from EA PCa (22Rv1, LNCaP, C4-2B, PC-3 and DU-145) and AA PCa (MDA PCa 2b) cell lines. Nuclei were visualized by counterstaining with DAPI (blue fluorescence). Merged images were acquired by overlapping DAPI, AR and SMARCD1 signals to visualize the colocalization (yellow) of AR and SMARCD1 either in nuclei or cytoplasm. All the captured fluorescent images were analyzed by using CellScans software V1.18. Five to six random images were captured by using 200× magnification.

FIGS. 4A-4D show immunofluorescence staining and western blot assays of the EA PCa and AA PCa cells treated with nonsense control (NC), miR-99b-5p mimic, Enz, or miR-99b-5p/Enz combination.

FIG. 4A shows that immunofluorescence showing mTOR (red fluorescence) and AR (green fluorescence) signals in EA PCa (22Rv1, LNCaP, C4-2B) and AA PCa (MDA PCa 2b) cell lines under treatments. Merged images were presented by overlaying AR/AR-V7 (green fluorescence), mTOR (red fluorescence) and DAPI (blue fluorescence) signals to visualize their cellular locations.

FIG. 4B shows immunofluorescence assays to examine the SMARCD1 (green fluorescence) and AR (red fluorescence) signals in EA PCa (22Rv1, LNCaP, C4-2B) and AA PCa (MDA PCa 2b) cell lines. The green and red fluorescence signals overlapped with blue fluorescence signal (DAPI) to visualize the cellular distribution of AR and SMARCD1 in EA and AA PCa cells under treatments. All the captured fluorescent images were analyzed by using CellScans software V1.18. Five to six random images were captured by using 200× magnification.

FIGS. 4C and 4D show western blot analysis of mTOR, pAR, AR/AR-V7 and SMARCD1 protein levels in cytoplasmic (FIG. 4C) and nuclear (FIG. 4D) fractions. GAPDH and Lamin B1 were used as endogenous controls for cytoplasmic and nuclear proteins, respectively. The cells were cultured followed by transfection with nonsense RNA or miR-99b-5p mimic. 24 h after the transfections, the PCa cells were treated with vehicle, or Enz (20 μM) for additional 48 h. NC, nonsense RNA with vehicle control; miR-99b-5p, miR-99b-5p mimic with vehicle; Enz, nonsense RNA with enzalutamide; miR-99b-5p/Enz, miR-99b-5p mimic with enzalutamide.

FIG. 5 shows immunofluorescence staining revealed the cellular localizations and expression levels of AR and mTOR in LNCaP.

FIG. 6 shows immunofluorescence staining revealed the cellular localizations and expression levels of AR and mTOR in 22Rv1.

FIG. 7 shows immunofluorescence staining revealed the cellular localizations and expression levels of AR and mTOR in C4-2B.

FIG. 8 shows immunofluorescence staining revealed the cellular localizations and expression levels of AR and mTOR in MDA PCa 2b.

FIG. 9 shows immunofluorescence staining revealed the cellular localizations and expression levels of AR and SMARCD1 in LNCaP.

FIG. 10 shows immunofluorescence staining revealed the cellular localizations and expression levels of AR and SMARCD1 in 22Rv1.

FIG. 11 shows immunofluorescence staining revealed the cellular localizations and expression levels of AR and SMARCD1 in C4-2B.

FIG. 12 shows immunofluorescence staining revealed the cellular localizations and expression levels of AR and SMARCD1 in MDA PCa 2b.

FIGS. 13A-13B show the western blot analysis of cytoplasmic (FIG. 13A) and nuclear (FIG. 13B) pAR/pAR-V7 and AR/AR-V7 levels in LNCaP, 22Rv1, C4-2B and MDA PCa 2b under different treatments in the absence or presence of androgen. GAPDH and Lamin B1 are endogenous cytoplasmic and nuclear controls, respectively. NC: nonsense RNA/vehicle, miR-99b-5p: miR-99b-5p mimic/vehicle, Enz: nonsense RNA/enzalutamide, miR-99b-5p/Enz: miR-99b-5p mimic/enzalutamide. Cytoplasmic and nuclear pAR/AR ratios were calculated by using the equation of (pAR density/AR density)/GAPDH density and (pAR density/AR density)/Lamin B1 density, respectively. The pAR/AR ratio of NC treated cells in androgen (-) was defined as 1.0.

FIGS. 14A-14D show in-vitro functional assays (TUNEL, MTT, and clonogenic assays) were performed in EA PCa (22Rv1, LNCaP, C4-2B) and AA PCa (MDA PCa 2b) cell lines treated with NC or miR-99b-5p mimic in presence or absence of 20 mM of Enz.

FIG. 14A shows TUNEL assays were carried out to visualize the DNA damages occurred during apoptotic events in PCa cell lines upon NC, miR-99b-5p, Enz, or miR-99b-5p/Enz treatments. Apoptotic events were detected based on the DNA damages (visualized as red fluorescent spots, defined as TUNEL-positive cells) in the nuclei (blue, DAPI staining). The red/purple signals derived from overlaying DAPI and TUNEL signals indicated the active apoptotic activities (DNA damages occurring in the nuclei) in the EA and AA PCa cells. For TUNEL, 3-4 random images were captured by using 100× magnification. Significantly different cell apoptotic capacities in miR-99b-5p vs. NC, Enz vs. NC or miR-99b-5p/Enz vs. NC (*p-value<0.05) were determined based on ANOVA with Dunnett's post-hoc test. Significantly different cell apoptotic capacities in miR-99b-5p/Enz vs. miR-99b-5p (#p-value<0.05) and miR-99b-5p/Enz vs. Enz (p-value<0.05) were determined based on ANOVA with Tukey's post-hoc test. Each value was represented as mean±SD (n=3).

FIGS. 14B and 14C show bright-field imaging (FIG. 14B) and MTT assays (FIG. 14C) of the EA (LNCaP, 22Rv1 and C4-2B) and AA (MDA PCa 2b) PCa organoids in response to NC, miR-99b-5p, Enz or miR-99b-5p/Enz treatments. Significantly different cell viabilities in miR-99b-5p vs. NC, Enz vs. NC or miR-99b-5p/Enz vs. NC (*p-value<0.05) were determined by MTT assays based on ANOVA with Dunnett's post-hoc test. Significantly different cell viability in miR-99b-5p/Enz vs. miR-99b-5p (#p-value<0.05) and miR-99b-5p/Enz vs. Enz (§ p-value<0.05) were determined based on ANOVA with Tukey's post-hoc test. Each value was determined by percentage of NC and each data point was represented as mean±SD (n=3-4). The volumes of organoids were calculated based on the equation: V=4/3 pR3, where V is volume and R is the radius averaged from 3-4 organoids. The volume of the NC treated organoid in each cell line was defined as 100%. Therefore, the relative organoid volume under treatment was determined by normalizing to its control (volume of miR99b-5p, Enz, or miR-99b-5p/Enz-treated organoid/volume of NC-treated organoid×100%). Significantly different organoid volume in miR-99b-5p/Enz vs. miR-99b-5p (#p-value<0.05) and miR-99b-5p/Enz vs. Enz (§ p-value<0.05) were determined based on ANOVA with Tukey's post-hoc test. Each value was represented as mean±SD (n=3-4).

FIG. 14D shows PCa colonies were counted and analyzed to evaluate the inhibitory effects by NC, miR-99b-5p, Enz or miR-99b-5p/Enz treatments. Significantly different colony forming capacities (*p-value<0.05, in miR-99b-5p vs. NC, Enz vs. NC, or miR-99b-5p/Enz vs. NC) were determined based on ANOVA with Dunnett's post-hoc test. Significantly different colony forming capacities in miR-99b-5p/Enz vs. miR-99b-5p (#p-value<0.05) and miR-99b-5p/Enz vs. Enz (@p-value<0.05) were determined based on ANOVA with Tukey's post-hoc test. Each value (% of NC-treated group) was represented as mean±SD (n=3-4). The 2D and 3D culture were grown followed by transfection with nonsense RNA or miR-99b-5p mimic. 24 h after the transfections, the PCa cells were treated with vehicle, or Enz (20 μM) for additional 48 h. NC, nonsense siRNA with vehicle control; miR-99b-5p, miR-99b-5p mimic with vehicle; Enz, nonsense RNA with enzalutamide; miR-99b-5p/Enz, miR-99b-5p mimic with enzalutamide.

FIGS. 15A-15B shows effects of NC, miR-99b-5p mimic, miR-99b-5p inhibitor, enzalutamide (Enz), and combination of miR-99b-5p mimic or inhibitor with Enz on cell viabilities of PCa cell lines.

FIG. 15A shows MTT assays of the EA (LNCaP, 22Rv1 and C4-2B) and AA (MDA PCa 2b) PCa cells in response to NC, miR-99b-5p mimic, Enz or miR-99b-5p mimic/Enz treatments.

FIG. 15B shows MTT assays of the EA (LNCaP, 22Rv1 and C4-2B) and AA (MDA PCa 2b) PCa cells in response to NC, miR-99b-5p inhibitor, Enz or miR-99b-5p inhibitor/Enz treatments. Significant difference on cell viabilities were recorded in miR-99b-5p mimic or inhibitor vs. NC, Enz vs. NC, or miR-99b-5p inhibitor/Enz vs. NC (*p-value, <0.05) and analyzed based on ANOVA with Dunnett's post-hoc test. Significantly different cell viability in miR-99b-5p mimic or inhibitor/Enz vs. miR-99b-5p mimic or inhibitor (#p-value<0.05), and miR-99b-5p mimic or inhibitor/Enz vs. Enz (@p-value<0.05) were determined based on ANOVA with Tukey's post-hoc test. Each value was determined by the percentage of NC and each data point was represented as mean±SD (n=6).

FIGS. 16A-16B shows morphological changes in EA and AA PCa cells treated with miR-99b-5p mimic, enzalutamide, and combination of miR-99b-5p mimic with enzalutamide.

FIG. 16A shows both panels were acquired by representative DAPI signals with blue and grey scales to evaluate the apparent changes in shape and/or morphology of nuclei in EA PCa (LNCaP, 22Rv1, and C4-2B) and AA PCa (MDA PCa 2b), in response to NC, miR-99b-5p, Enz or miR-99b-5p/Enz treatments. Both (DAPI and grey-scaled) images show the morphological changes in nuclei as blebbing, invagination, or herniation under different (miR-99b-5p, Enz or miR-99b-5p/Enz) treatments, compared to NC groups.

FIG. 16B morphological changes of EA PCa (LNCaP, 22Rv1, and C4-2B) and AA PCa (MDA PCa 2b), in response to NC, miR-99b-5p, Enz or miR-99b-5p/Enz treatments. Cell images were captured from 3-4 random areas at 20× magnification by using CellSens V1.18 software (Olympus, Waltham, MA, USA).

FIGS. 17A-17B show ChIP-qPCR assays for examining the binding affinities of mTOR to the mTOR target genes in EA and AA PCa cells under treatments of NC, miR-99b-5p, Enz, and miR-99b-5p/Enz. The mTOR ChIP assays were followed by qPCR assays of KLK3 (FIG. 17A), ENO1 (FIG. 17A), SLC26A3 (FIG. 17B), and TMPRSS2 (FIG. 17B) (genes co-targeted by mTOR and AR). Significant differences of mTOR occupancies (*p-value<0.05 in miR-99b-5p vs. NC, and #p-value<0.05 in Enz vs. NC) on the target genes were determined based on ANOVA with Dunnett's post-hoc test. Whereas, significant differences of mTOR occupancies in miR-99b-5p/Enz vs. miR-99b-5p and miR-99b-5p/Enz vs. Enz (§ p-value<0.05) on the target genes were determined based on ANOVA with Tukey's post-hoc test. Each value was determined by the ratio of ChIP signal/Input signal in qPCR reactions, and each data was derived from mean±SD (n=4). The PCa cells were cultured followed by transfection with nonsense RNA or miR-99b-5p mimic. 24 h after the transfections, the PCa cells were treated with vehicle, or Enz (20 μM) for additional 48 h. NC, nonsense siRNA with vehicle control; miR-99b-5p, miR-99b-5p mimic with vehicle; Enz, nonsense RNA with enzalutamide; miR-99b-5p/Enz, miR-99b-5p mimic with enzalutamide.

FIGS. 18A-18B show proposed model of miR-99b-5p mediated mTOR/AR/SMARCD1 signaling axis involved in CRPC development/progression and/or AA PCa aggressiveness.

FIG. 18A shows downregulation of miR-99b-5p in PCa increases mTOR, AR and SMARCD1 protein levels and promotes the translocation of mTOR/AR/SMARCD1 complex to nucleus, consequently inducing metabolic reprogramming and triggering the development of CRPC or promoting the AA PCa aggressiveness.

FIG. 18B shows an overexpression of miR-99b-5p as a therapeutic strategy for treating aggressive CRPC and AA PCa. Tumor suppressor miR-99b-5p inhibits protein expression of mTOR, AR and SMARCD1, blocks mTOR/AR/SMARCD1 translocation to nucleus and restores the metabolic gene program. Combining miR-99b-5p with Enz synergistically enhances the cell apoptosis and inhibits cell proliferation/survival in refractory CRPC and aggressive AA PCa.

FIGS. 19A-19D show immunofluorescence revealed an opposite effect of miR-99b-5p mimic and/or Enz/Abi on regulating E-cadherin and N-Cadherin expression in AA PCa and EA PCa cells. Immunofluorescence assays were performed to examine the protein levels of E-Cadherin (red fluorescence) and N-Cadherin (green fluorescence) signals in LNCaP (FIG. 19A), 22Rv1 (FIG. 19B), C4-2B (FIG. 19C), and MDA PCa 2b cells (FIG. 19D) treated with NC, miR-99b-5p mimic, Enz, Abi, miR-99b-5p/Enz, and miR-99b-5p/Abi. Merged images were obtained by overlaying E-Cadherin and N-Cadherin signals to reveal the subcellular localization of these two proteins (i.e., yellow signal indicates the colocalization of E-Cadherin and N-Cadherin in cytoplasm). These images are presented as representative fluorescence staining images from 3-4 independent experiments.

FIGS. 20A-20D shows immunofluorescence staining assays of Vimentin and Snail proteins in AA and EA PCa cells under different treatments. Immunofluorescence assays were performed to examine the protein levels of Snail (red fluorescence) and Vimentin (green fluorescence) signals in LNCaP (FIG. 20A), 22Rv1 (FIG. 20B), C4-2B (FIG. 20C), and MDA PCa (FIG. 20D) 2b cells treated with NC, miR-99b-5p mimic, Enz, Abi, miR-99b-5p/Enz, and miR-99b-5p/Abi. Merged images were obtained by overlaying E-Cadherin and N-Cadherin signals to reveal the subcellular localization of these two proteins (i.e., yellow signals indicate the colocalization of Snail and Vimentin proteins in cytoplasm). These images are presented as representative fluorescence staining images from 3-4 independent experiments.

FIGS. 21A-21B show western blot analyses of the EMT markers E-cadherin, N-cadherin, Snail, and Vimentin proteins in EA and AA PCa cells under different treatments. FIG. 21A shows a Western blot analysis of mTOR and AR levels upon transfection of miR-99b-5p mimic in EA and AA PCa cells. FIG. 21B show western blot analyses of N-cadherin, E-cadherin, Snail, and Vimentin proteins in PCa cells under treatments of NC, miR-99b-5p, Enz, Abi, miR-99b-5p/Enz, and miR-99b-5p/Abi. 3-actin was used as an endogenous control. The Western blot images are representative images selected from 3 independent Western blot experiments. The uncropped blots are shown in the Supplementary Materials.

FIGS. 22A-22B shows wound-healing assays to determine migration of EA PCa cell lines (LNCaP, 22Rv1, and C4-2B) and AA PCa cell lines (MDA PCa 2b) transfected with NS or miR-99b-5p mimic in the absence or presence of 20 μM Enz or 10 μM Abi. FIG. 22A shows Representative phase-contrast microscope images showing the areas covered by the PCa cells (with different treatments) at 0 and 48 h. FIG. 22B shows bar graphs showing migration rates of the PCa cells (under different treatments) within 48 h. The migration rate was determined within 48 h using a wound-healing assay (described in Materials and Methods). Significantly different migration rates were determined: *p-value<0.05 in either treatment vs. NC group, #p-value<0.05 in miR-99b-5p/Enz (or miR-99b/Abi) vs. miR-99b-5p treated cells, § p-value<0.05 in miR-99b-5p/Enz vs. Enz-treated cells, and @p-value<0.05 in miR-99b-5p/Abi vs. Abi-treated cells. The migration rate of the NC treated cells was defined as 100% for data normalization across different treatment groups, and p-values were determined using ANOVA with Tukey's post hoc test. Each value was derived from the mean±SD (n=3-4).

FIGS. 23A-23D show transwell-mediated migration assays of the PCa cells treated with NC, miR-99b-5p mimic, Enz, Abi, miR-99b-5p/Enz, or miR-99b-5p/Abi. Bar graph showing the migration capacities of NC-, miR-99b-5p-, Enz-, Abi-, miR-99b-5p/Enz-, and miR-99b-5p/Abi-treated LNCaP (FIG. 23A), 22Rv1 (FIG. 23B), C4-2B (FIG. 23C), and MDA PCa 2b (FIG. 23D) cells. Note the migration capacities were defined by % of migrated cells through transwell assays. Significantly different migration capacities (defined as % migrated cells) were determined: *p-value<0.05 in either treatment vs. NC group, #p-value<0.05 in miR-99b-5p/Enz (or miR-99b/Abi) vs. miR-99b-5p treated cells, § p-value<0.05 in miR-99b-5p/Enz vs. Enz-treated cells, and @p-value<0.05 in miR-99b-5p/Abi vs. Abi-treated cells. The migration rate of the NC-treated cells was defined as 100% for data normalization across different treatment groups, and p-values were determined based on ANOVA with Tukey's post hoc test. Each value was represented as the mean±SD (n=3).

FIGS. 24A-24B show cell adhesion assays of the PCa cells under treatments of NC, miR-99b-5p mimic, Enz, Abi, miR-99b-5p/Enz, and miR-99b-5p/Abi. FIG. 24A show representative images showing adhesion capacities of EA and AA PCa cells to extracellular matrix (ECM) in response to treatments of NC, miR-99b-5p mimic, Enz, Abi, miR-99b-5p/Enz, and miR-99b-5p/Abi. FIG. 24B show bar graphs quantifying the cell adhesion capacities (to ECM) of the PCa cells under different treatments. Significantly different cell-EMC adhesion capacities were determined: *p-value<0.05 in either treatment vs. NC group, #p-value<0.05 in miR-99b-5p/Enz (or miR-99b/Abi) vs. miR-99b-5p treated cells, § p-value<0.05 in miR-99b-5p/Enz vs. Enz-treated cells, and @p-value<0.05 in miR-99b-5p/Abi vs. Abi-treated cells. The migration rate of the NC treated cells was defined as 100% for data normalization across different treatment groups, and p-values were measured using ANOVA with Tukey's post hoc test. Each data value was taken from the mean±SD (n=3).

FIG. 25 captures representative phase-contrast microscope images showing the capillary-like tube formation of HUVEC cells incubated with condition medium derived from PCa cells under different treatments. The images were captured using an inverted phase-contrast microscope, and the representative images were selected from 3-4 areas of at least two independent tube formation assays per cell treatment group.

FIG. 26 shows a graphical representation of miR-99b-5p-mediated suppression of EMT in AA PCa and CRPC. As illustrated, miR-99b-5p targets and inhibits the expression of MTOR, AR, and VEGFA, consequently suppressing the AR-, mTOR/RhoA/Rac1-, VEGF-, and HIF-1α-mediated activation of EMT.

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

Materials and Methods for a Combination Therapy of miR-99b-5p and Androgen Receptor Antagonists for Treating Castration-Resistant Prostate Cancer

### Monolayer and Organoid Cell Cultures

The human PCa cell lines PC-3, DU-145, LNCaP, 22Rv1, C4-2B and MDA PCa 2b were used in the present disclosure. Specifically, PC-3 and DU-145 are AR-negative EA PCa cell lines derived from bone and brain metastasis, respectively. LNCaP represents an androgen-dependent EA PCa cell line derived from lymph node. 22Rv1 and C4-2B represent castration-resistant prostate cancer (CRPC) cell lines derived from EA PCa patients, while MDA PCa 2b represents an androgen-independent AA PCa cell lines derived from bone metastasis. All PCa cell lines, including LNCaP (ATCC CRL-1740), PC-3 (ATCC CRL-1435), DU145 (ATCC HTB-81), 22Rv1 (ATCC CRL-2505), C4-2B (ATCC CRL-3315) and MDA PCa 2b (ATCC CRL-2422), were purchased from American Type Culture Collection (ATCC, Manassas, VA, USA). LNCaP and 22Rv1 were cultured in RPMI-1640 with 10% fetal bovine serum (FBS). DU-145 and PC-3 were cultured in DMEM with 10% FBS. C4-2B was grown in advanced DMEM with 10% FBS, and MDA PCa 2b was cultured in BRFF-HPC1 with 20% FBS.

For establishing PCa organoids, the PCa cells (LNCaP, PC-3, DU-145, 22Rv1, C4-2B and MDA PCa 2b) were grown to 80% confluency, and washed three times with 1×PBS then treated with 0.25% trypsin-EDTA at 37° C. Thereafter, the cell suspension was centrifuged at 150×g at 4° C. for 5 min, and resuspended in advanced DMEM/F12 media then mixed with pre-thawed matrigel (Corning Life Sciences, Tewksbury, MA, USA) and dropped at the middle of the well of a pre-warmed culture plate. Then, cell culture plates were placed at upside down in the incubator with 5% CO2 at 37° C. for 15 min to allow the matrigel to solidify into a dome. After 15 min, the pre-warmed organoid medium (diluted B27, 1.25 mM N-acetyl-L-cysteine, 5 ng/ml EGF, 100 ng/ml Noggin, 500 ng/ml recombinant R-spondin 1, 500 nM A83-01, 10 ng/ml FGF10, 5 ng/ml FGF2, 1 μM PGE2, 10 mM nicotinamide, 10 μM SB202190, 1 nM DHT, and 10 μM Y-27632 dihydrochloride) was added and the cells were grown in 5% CO2 at 37° C. for 14 days. These cell lines and organoids were served as an in vitro model to examine the functional action of miR-99b-5p/MTOR/AR pairing and Enz in both EA PCa and AA PCa cell lines and their derived organoid.

### MicroRNA Transfection and Drug Treatment in EA and AA PCa Cell Models

To examine the inhibitory effects of miR-99b-5p and Enz treatments in EA and AA PCa, the EA PCa LNCaP, EA CRPC (22Rv1 and C4-2B) and AA PCa MDA PCa 2b cell lines were seeded at a density of 3×10⁵ cells/well in 6-well plates. The PCa cells (2D or 3D cultures) were grown for 24 h followed by the transfection with nonsense RNA or miR-99b-5p mimic (Ambion, Austin, TX, USA) using DharmaFECT4 transfection reagent (Dharmacon, Lafayette, CO, USA). After transfection for 24 h, the PCa cells were treated with either vehicle or enzalutamide for additional 48 h. The four treatment groups were designated as NC (nonsense control, referring to nonsense RNA with vehicle control), miR-99b-5p (miR-99b-5p mimic with vehicle), Enz (nonsense RNA with 20 mM enzalutamide), and miR-99b-5p/Enz (miR-99b-5p mimic with 20 mM enzalutamide). 20 mM of Enzalutamide concentration was used in the experiments, according to previous studies.

### Western Blot Analysis

The western blot assays were performed using standardized protocol established as previously described. Briefly, the PCa cells were collected and total proteins were extracted using M-PER Mammalian Protein Extraction Reagent (Thermo Fisher Scientific, Waltham, MA, USA) with protease and phosphatase inhibitor cocktail (Thermo Fisher Scientific, Waltham, MA, USA). Quantification of protein concentrations from individual protein samples were determined using BCA assay kit (Thermo Fisher Scientific, Waltham, MA, USA). Bolt 4-12% Bis-Tris mini protein gels (Thermo Fisher Scientific, Waltham, MA, USA) were used for running protein gel electrophoresis. The primary antibodies used in the present disclosure were rabbit monoclonal antibodies against AR/AR-V7 (catalog #5153, 1:1000), pAR (catalog #16969, 1:1000), mTOR (catalog #2972, 1:1000), GAPDH (catalog #5174, 1:1000), and Lamin B1 (catalog #13435, 1:1000) from Cell Signaling Technology (Danvers, MA, USA), and polyclonal rabbit antibody against SMARCD1 from Invitrogen (catalog #PA5-30175, 1:1000, Waltham, MA, USA). The secondary antibody used were anti-rabbit IgG-HRP (catalog #4030-05, 1:10000) and anti-mouse IgG-HRP (catalog #1033-05, 1:10000) antibodies purchased from Thermo Fisher Scientific (Waltham, MA, USA).

### Immunofluorescence Staining for 2D Monolayer and 3D Cultures

For this procedure, 4×10⁴ cells were seeded on cover slip and allowed to attach for 24 h in 5% CO2 incubator at 37° C. All the experimental cells were subjected to immunofluorescence assays 48 h following transfection of NC or miR-99b-5p mimic, and/or Enz treatment. Briefly, cells were washed with 1×PBS, fixed in 4% paraformaldehyde, and permeabilized with 0.1% Triton X-100. Cells were then blocked for 1 h with 2% BSA in 1×PBS. Primary antibodies against mTOR (catalog #2972, Cell Signaling Technology, Waltham, MA, USA, or catalog #sc-517464, Santa Cruz Biotechnology, Dallas, TX, USA), AR (catalog #5153, from Cell Signaling Technology, Waltham, MA, USA, or catalog #sc-7305, Santa Cruz Biotechnology, Dallas, TX, USA), SMARCD1 (catalog #PA5-30175, Invitrogen, Waltham, MA, USA) and were applied to the fixed/permeabilized cells for incubation overnight at 4° C. The cells were washed twice with 1×PBS and followed by the incubation of Alexa-Fluor-488-conjugated anti-rabbit and Alexa-Fluor-594-conjugated anti-mouse antibodies, respectively (catalog #A32731 and #A32744, from Invitrogen, Waltham, MA, USA) for 1 h at room temperature. Thereafter, cells were washed twice with 1×PBS for 5 min and the nuclei were visualized by staining with DAPI from Invitrogen (catalog #P36981, Waltham, MA, USA). The fluorescence-labeled cells were mounted on glass slides and visualized by fluorescence microscopy (Olympus, Waltham, MA, USA). Cell images were captured from 3-4 random areas at 20× magnification by using CellSens V1.18 software (Olympus, Waltham, MA, USA).

For 3D immunofluorescence staining procedure: media was removed and the matrigel domes were washed with 1×PBS, fixed in 4% paraformaldehyde for 30-60 min at room temperature. After fixation, the matrigel was dissolved, and the domes were detached to release organoids from matrigel by using differential centrifugation process. The resulting pellet was then suspended in 1×PBS and the suspension containing organoids was spread on the cover slips followed by incubation for 15-20 min to evaporate the 1×PBS at 37° C. Thereafter, the organoids were permeabilized, blocked, labeled by primary antibodies and secondary antibodies conjugated with fluorescence, mounted in presence of DAPI, and visualized under fluorescence microscope using the same protocol described for the 2D cultures. Images were captured from 3-4 random areas at 20× magnification by using CellSens V1.18 software (Olympus, Waltham, MA, USA).

TdT-Mediated dUTP-Biotin End-Labeling Assays

TUNEL assay was performed as per the manufacturer's protocol (Click-iT™ Plus In-situ Apoptosis Detection with Alexa Fluor Dyes, Thermo Fisher Scientific, Waltham, MA, USA). Specifically, 4×10⁴ cells were seeded on coverslips and allowed to attach for 24 h in 5% CO2 incubator at 37° C. After treatment of NC RNA, miR-99b-5p mimic, and/or Enz for 48 h, cells were fixed in 4% paraformaldehyde and washed three times with 1×PBS followed by the incubation with 0.25% Triton X-100 in 1×PBS and conclusively the cells were incubated with differential steps of TUNEL reaction mixture as per the manufacturer's protocol. At the end of the reaction, the processed cells were counterstained with DAPI for 5 min, at room temperature in the dark. All the fluorescence-labeled cells were mounted on glass slides and visualized by Olympus BX3 fluorescence microscope (Olympus, Waltham, MA, USA). Cell images were captured from 3-4 random areas at 10× magnification by using CellSens V1.18 software (Olympus, Waltham, MA, USA).

### Cell Viability Assays for PCa Organoid Cultures

After miRNA transfection and/or drug treatment regimen, organoid formation/growth was captured by brightfield or phase contrast microscopy. In addition, the cell viabilities of the PCa organoids were assessed by 3-(4,5-dimethylthiazol-2-yl)-2,5 diphenyl tetrazolium bromide (MTT) solution. Briefly, 0.5 mg/ml MTT was added to the organoid cultures and incubated at 37° C. for 3 h. The matrigel containing the PCa organoids was solubilize in a 2% sodium dodecyl sulfate solution for 2 h. Thereafter, 100 ml DMSO was added for 1 h to solubilize the reduced MTT or tetrazolium salt crystals, and the absorbance at the wavelength of 570 nm was detected using Mutiskan FC microplate photometer (Thermo Fisher Scientific Waltham, MA, USA). The cell viabilities of the organoids were presented as percentages of absorbance values from the controls. The data were analyzed using the Prism 9 program (GraphPad Software, La Jolla, CA) for graphing and statistical analysis.

### Colony Forming (Clonogenic) Assay

The EA PCa LNCaP, EA CRPC (22Rv1 and C4-2B), and AA PCa (MDA PCa 2b) cells were seeded at a density of 500 cells/well in triplicate formats. After 24 h, cells were transfected and/or treated for 24 h and kept at 37° C. in 5% CO₂ for 14 days. The media was replaced every 3 days. At the end of experiment, cells were fixed with 90% chilled methanol followed by staining with 0.5% crystal violet. The colonies were counted and scored as percentage of the control experimental group.

### Chromatin Immunoprecipitation Assay

First, the PCa cell lines LNCaP, 22Rv1, C4-2B, and MDA PCa 2b were treated with nonsense RNA, miR-99b-5p, and/or Enz. After 48 h, the cells were harvested and subjected to ChIP assays using ChIP assay kit (Millipore, Burlington, MA, USA) according to the manufacture's protocol. Briefly, 1×10⁶ cells were treated with 1% formaldehyde for 15-20 min at room temperature, to crosslink the proteins with DNA. After that, the cells were washed twice with 1×PBS, collected by scraping cells from the plates, then resuspended in SDS lysis buffer for 10 min on ice. The cell lysates were sonicated for shearing DNA to lengths between 250 and 750 bp, then the lysates were centrifuged at 1,4000×g for 20 min at 4° C. Thereafter, the anti-mTOR antibody was added to immunoprecipitate the mTOR/DNA complexes at 4° C. for 1 h with agitation. 50 ml of protein A agarose/salmon sperm DNA slurry was then added for incubation at 4° C. overnight. After incubation, the solution was spun down and the agarose/antibody/protein-DNA complexes were washed by low salt, high salt and LiCl immune complex wash buffers, and TE buffer, respectively. The agarose/antibody/protein-DNA complexes were then added with 8 ml of 5M NaCl and incubated at 65° C. for 8 h to reverse the DNA/protein crosslinks. The Input and ChIP samples were then subjected to qPCR reactions to check the mTOR occupancies (determined by the ratios of mTOR ChIP/total Input) on KLK3, ENO1, SLC26A3 and TMPRSS2 genes. The primer sequences were listed as follows: KLK3-forward: 5′-TGCCACTGGTGAGAAACCTGAGAT-3′ (SEQ ID NO: 1), KLK3-reverse: 5′-TCAGAGACAAAGGCTGAGCAGGTT-3′ (SEQ ID NO: 2), ENO1-forward: 5′-GGGGCTTATGTCTTGCCAGT-3′ (SEQ ID NO: 3), ENO1-reverse: 5′-AGAGGTTTCCATTGGTTACTTGGT-3′ (SEQ ID NO: 4), SLC26A3-forward: 5′-AGGATGTGGGCATCTTTGGG-3′ (SEQ ID NO: 5), SLC26A3-reverse: 5′-GAGATAGCAGCCAGGCACAA-3′ (SEQ ID NO: 6), TMPRSS2-forward: TGAGACTAGCCTGGACACCA-3′ (SEQ ID NO: 7), and TMPRSS2-reverse: 5′-AAGCTCACTGCAGCCTCAAA-3′ (SEQ ID NO: 8).

### Statistical Analysis

Results were expressed as mean±standard deviation (SD). All data were analyzed by using analysis of variance (ANOVA) followed by Tukey's test. Values of p<0.05 were considered as significant. All the statistical analyses were performed using GraphPad Prism 9.0 (Graph Pad Software, Inc., San Diego, CA, USA).

Results for a Combination Therapy of miR-99b-5p and Androgen Receptor Antagonists for Treating Castration-Resistant Prostate Cancer
Subcellular Localizations of AR, AR-V7, mTOR and SMARCD in EA and AA PCa Cells

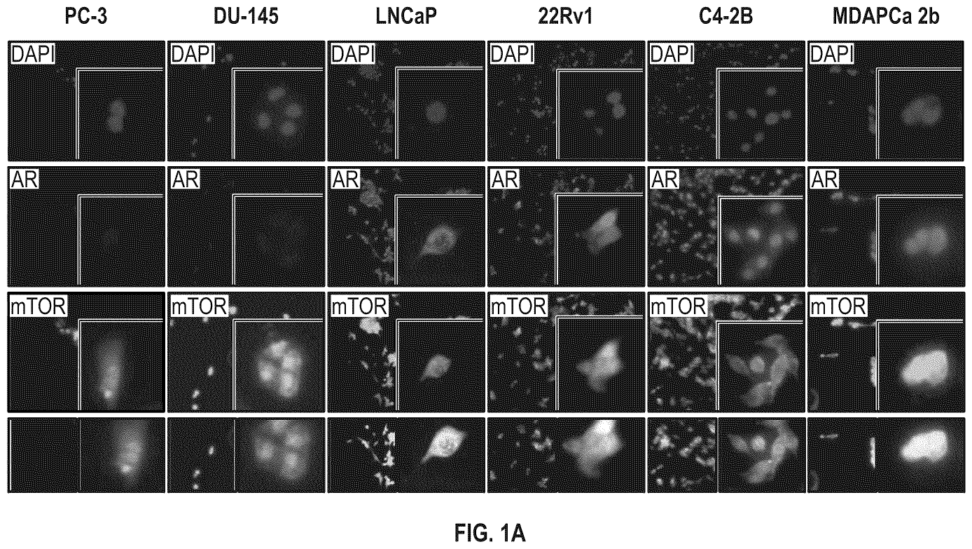

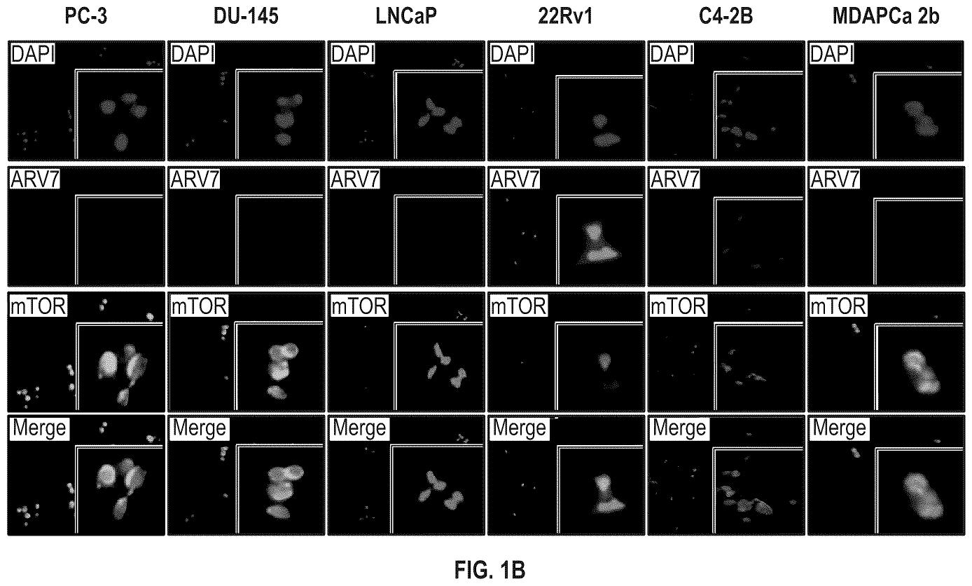

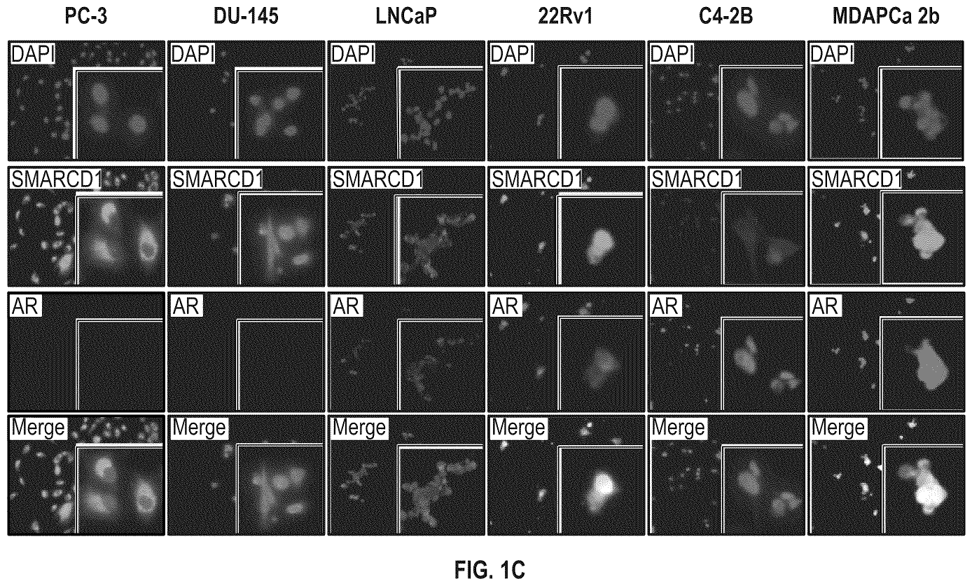

To understand the regulation of miR-99b-5p/mTOR/AR signaling, the cellular locations of AR, AR-V7, mTOR and SMARCD1 were explored in EA PCa (LNCaP, PC-3, DU-145, 22Rv1, and C4-2B) and AA PCa (MDA PCa 2b) cell lines. LNCaP is an androgen-sensitive PCa, PC-3 and DU-145 are AR-negative cell lines derived from bone and brain metastases, while C4-2B (derived from parental line LNCaP) and 22Rv1 represented CRPC cell lines from EA. MDA PCa 2b is an AR-mutated and androgen-independent AA PCa derived from the bone metastasis. The immunofluorescence results confirmed that there was no AR expression in PC-3 or DU-145, whereas an abundant AR expression level was observed in LNCaP, 22Rv1, C4-2B and MDA PCa 2b (green fluorescence signals in FIG. 1A). Notably, CRPC 22Rv1, C4-2B and AA PCa MDA PCa 2b demonstrated significantly higher expression levels of nuclear AR, compared to LNCaP that predominately expressed cytoplasmic AR (green fluorescence and merged images in FIG. 1A). In contrast, mTOR protein was generally expressed in cytoplasm and nuclei of all the EA and AA PCa cell lines (red fluorescence signals in FIG. 1A). The EA CRPC line, 22Rv1, particularly expressed a high level of nuclear AR-V7 splice isoform (green fluorescence signals in FIG. 1B). It was further examined the expression profiles of SMARCD1, an AR coactivator, in these six PCa cell lines. The immunofluorescence assays have revealed that SMARCD1 was not only expressed in cytoplasm of all PCa cell lines, but was expressed in the nuclear fractions in EA CRPC line 22Rv1 and AA PCa line MDA PCa 2b (green fluorescence in FIG. 1C).

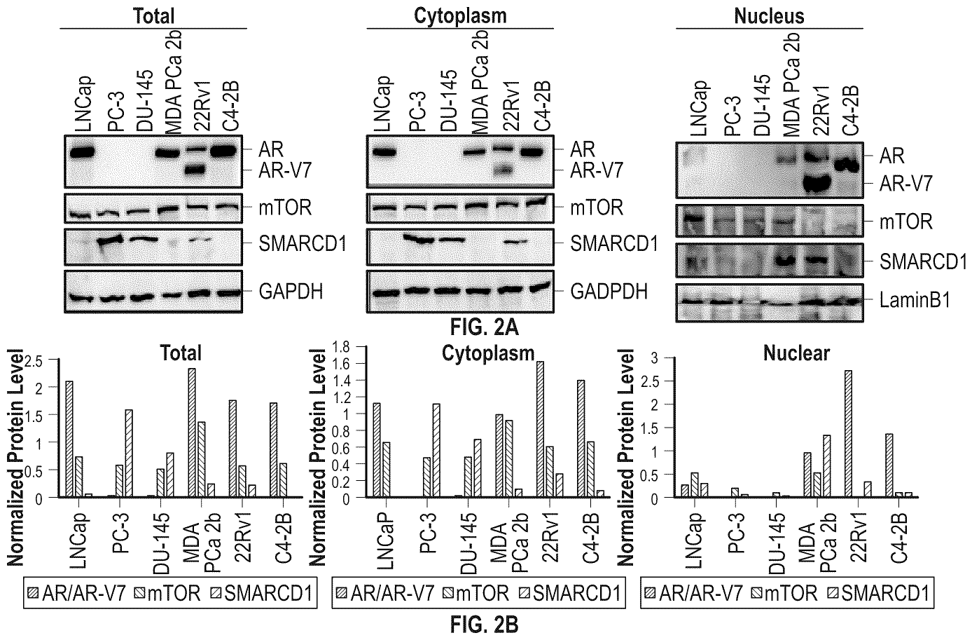

Next, western blot assays were conducted to verify the total, cytoplasmic and nuclear protein levels of AR, AR-V7, mTOR, and SMARCD1 in these six PCa cell lines. Consistent with the expression patterns shown in the immunofluorescence assays, AR was expressed in LNCaP, 22Rv1, C4-2B and MDA PCa 2b, mTOR was comparably expressed in all PCa cell lines, and differential expression levels of SMARCD1 were observed in different cell lines (FIGS. 2A-2B). Interestingly, nuclear AR/AR-V7 and/or nuclear SMARCD1 were expressed in either EA CRPC (22Rv1, C4-2B) or AA PCa (MDA PCa 2b). Nuclear mTOR, a more oncogenic form of mTOR, was highly expressed in MDA PCa 2b cells (FIGS. 2A-2B). Taken together, nuclear AR/AR-V7, SMARCD1 and mTOR (i.e. active/phosphorylated forms of proteins) were enriched in androgen-independent AA PCa (MDA PCA 2b), and nuclear AR/AR-V7 and SMARCD1 were enriched in EA CRPC (22Rv1 and C4-2B), potentially explaining the aggressiveness of AA PCa and CRPC.

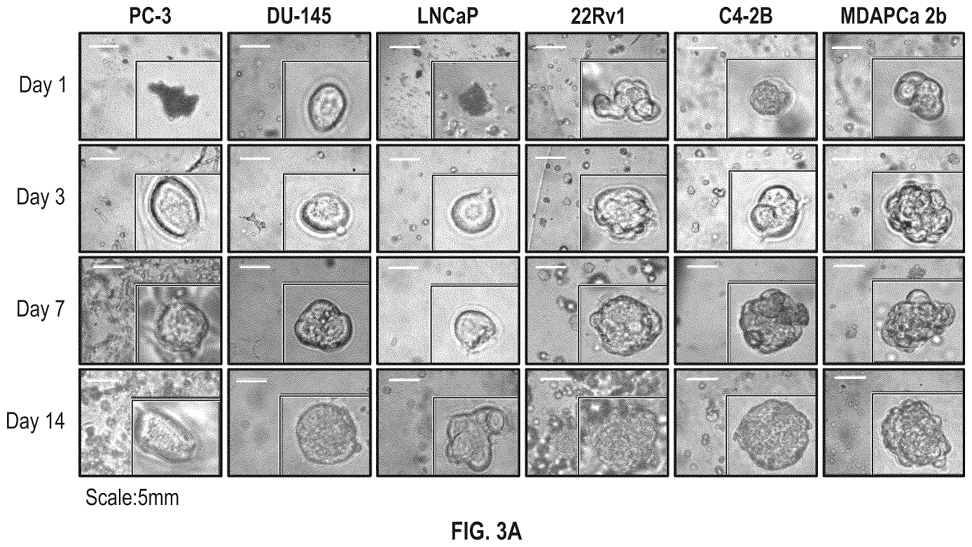

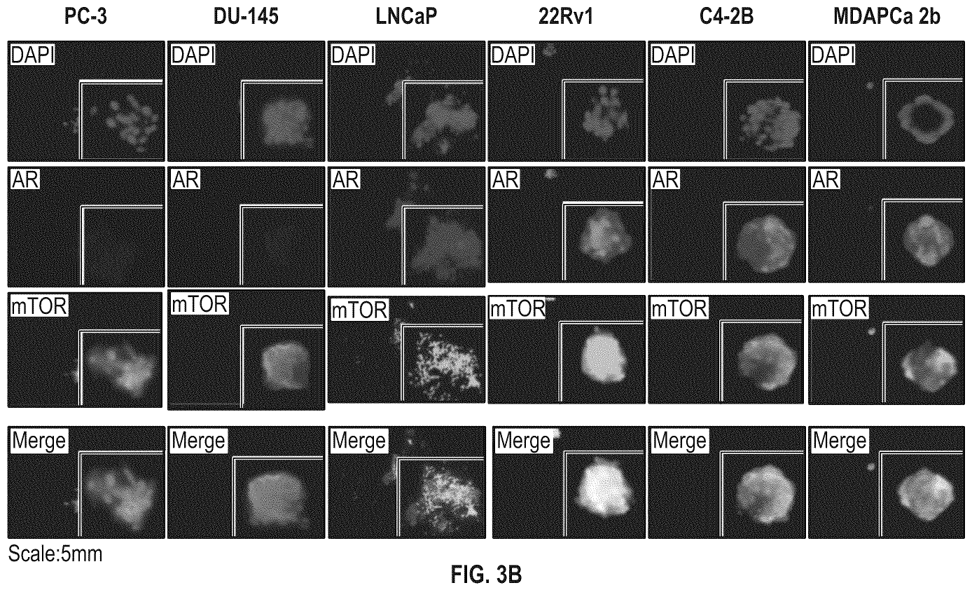

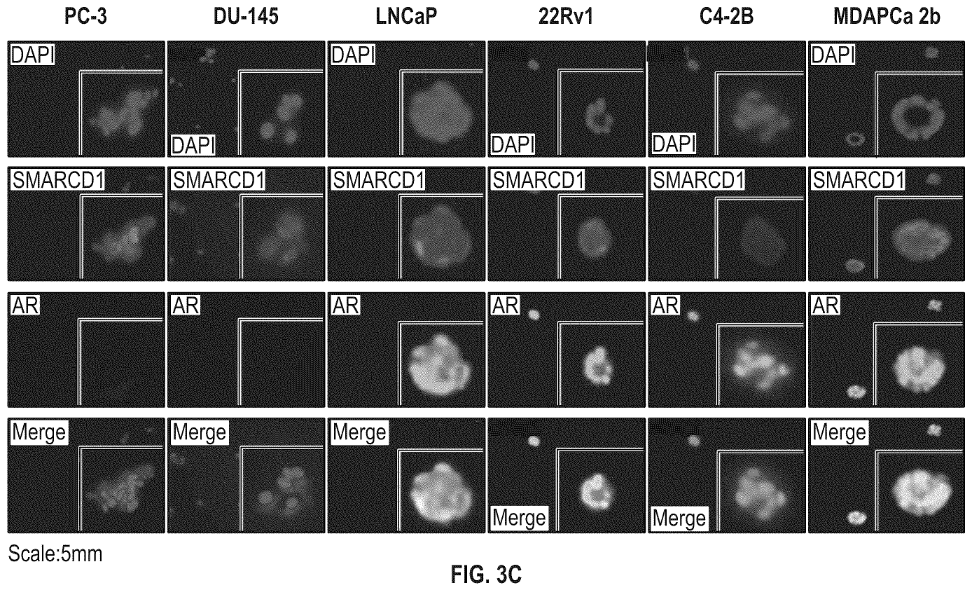

To further examine the expression levels/patterns and functional implications of AR/AR-V7, mTOR and SMARCD1 in consideration of tumor microenvironment, the 3D cultures were developed from PC-3, DU-145, LNCaP, 22Rv1, C4-2B and MDA PCa 2b cells. As shown in FIG. 3A, all the cell lines were grown in the matrigels and the volumes of the PCa organoids were consistently increased in a time dependent manner. At the initial stage of organoid culture (day 1), all the experimental PCa cells prominently exhibited mono- or bi-layer growing patterns in matrigels, with 2-4 cells aggregated together. On day 3 and 7, organoid cultures were gradually formed, evident from the aggregation of dozens of cells. On day 14, the organoids of the PCa cells were established, and the organoids were subjected to immunofluorescence assays for visualizing the expression levels/patterns of AR/AR-V7, mTOR, SMARCD1. Similar to the expression profiles shown in the 2D (mono layer) cultures, AR was not expressed in PC-3 and DU-145 but highly expressed in LNCaP, 22Rv1, C4-2B and MDA PCa 2b organoids (green fluorescence, FIG. 3B). Notably, higher nuclear AR levels were observed in EA CRPC (22Rv1 and C4-2B) and AA PCa (MDA PCa 2b) organoids (FIGS. 3B, C). The AR co-activator SMARCD1 was predominately expressed in cytoplasm of PC-3 and DU-145. In contrast, nuclear SMARCD1 was particularly enriched in EA CRPC 22Rv1 and AA PCa MDA PCa 2b cells (FIG. 3C). These results, again, showed a generalized consistency with the results from western blot assays (FIGS. 2A-2B).

Transfection of miR-99b-5p Mimic and Treatment of Enz Inhibit the Expression Levels and/or Nuclear Translocation of mTOR, SMARCD1 and AR in EA and AA PCa Cells

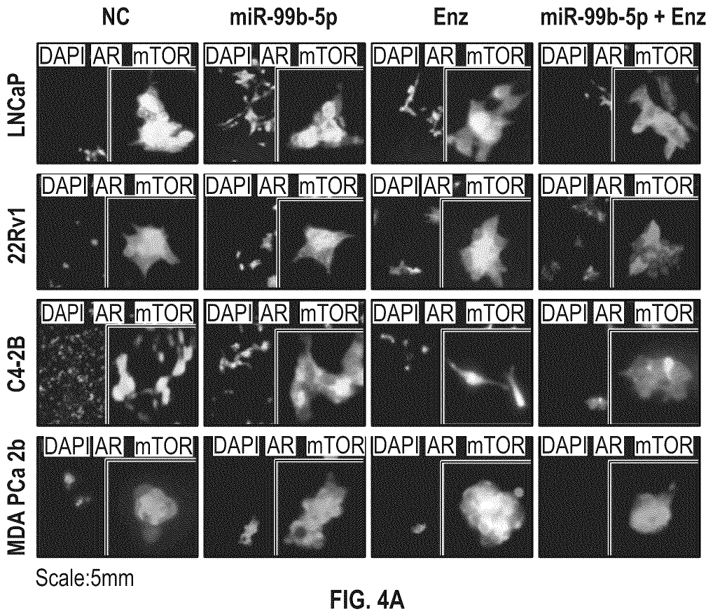

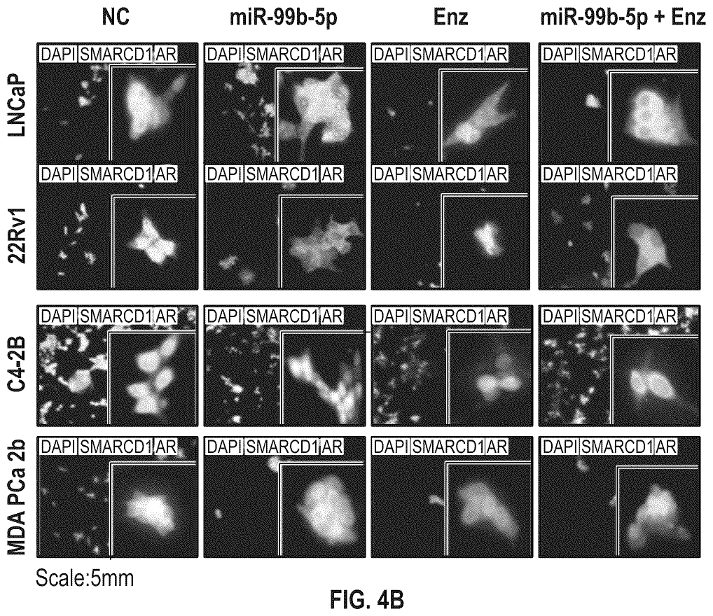

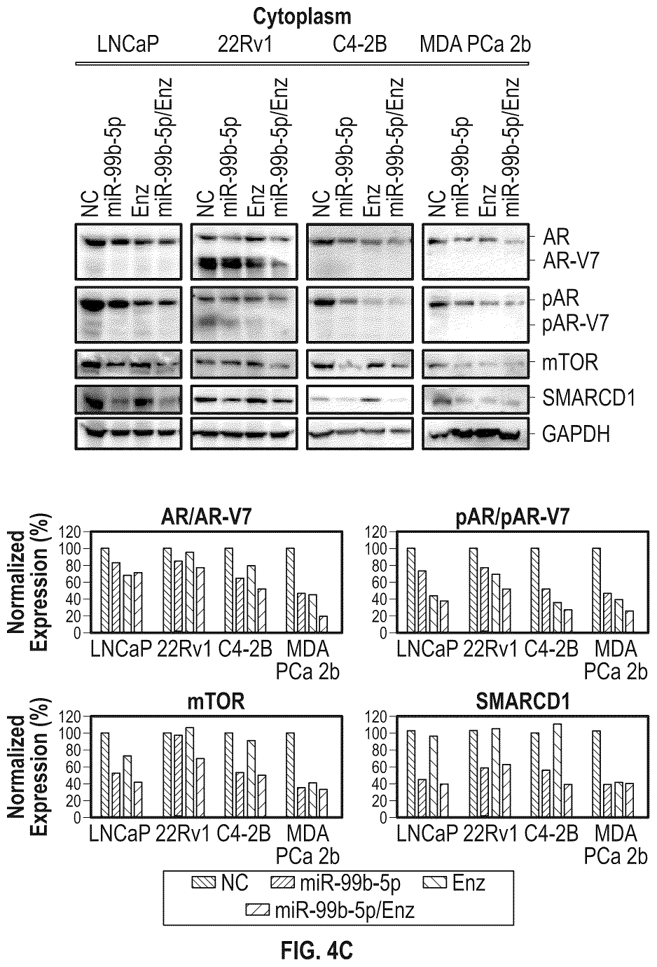

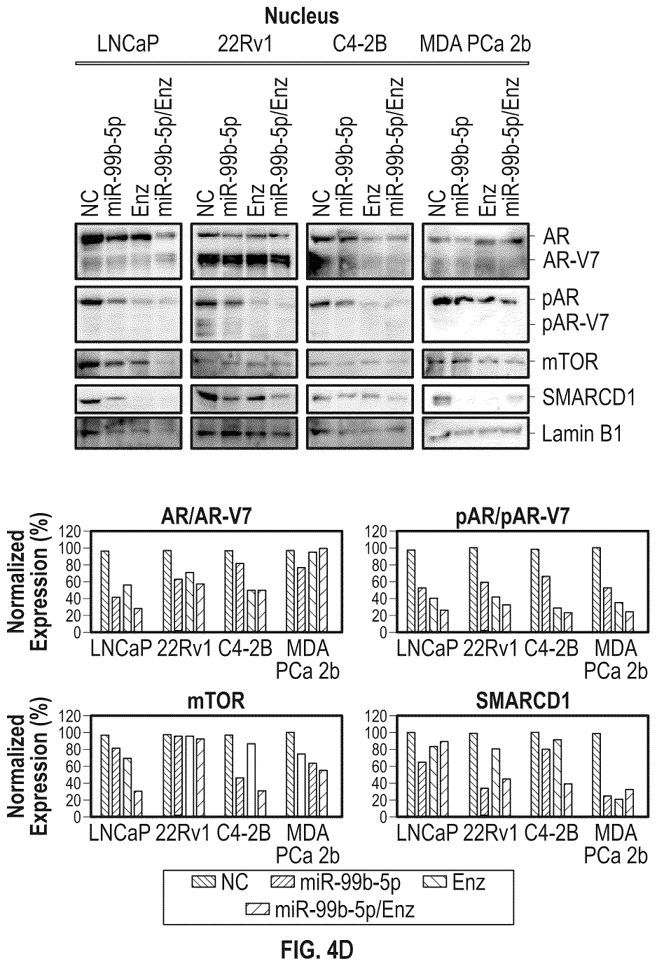

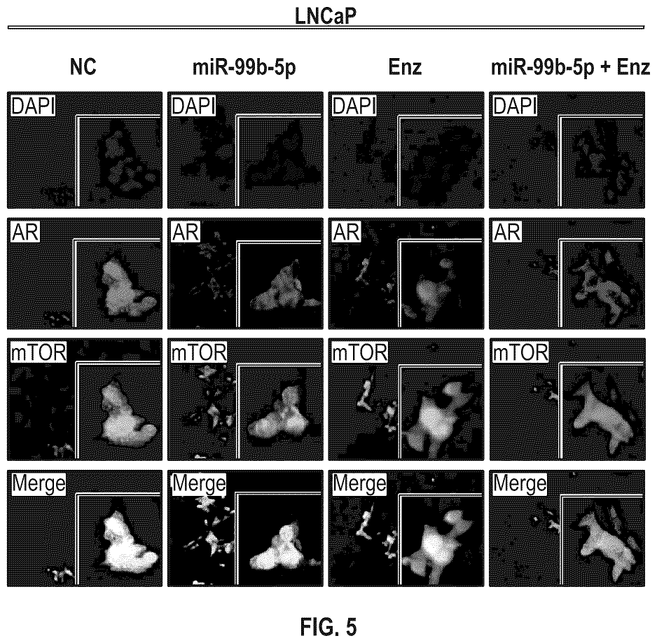

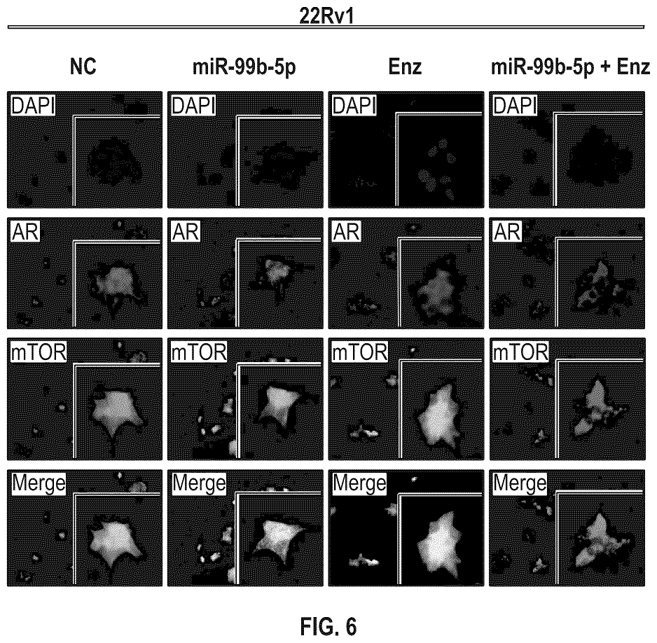

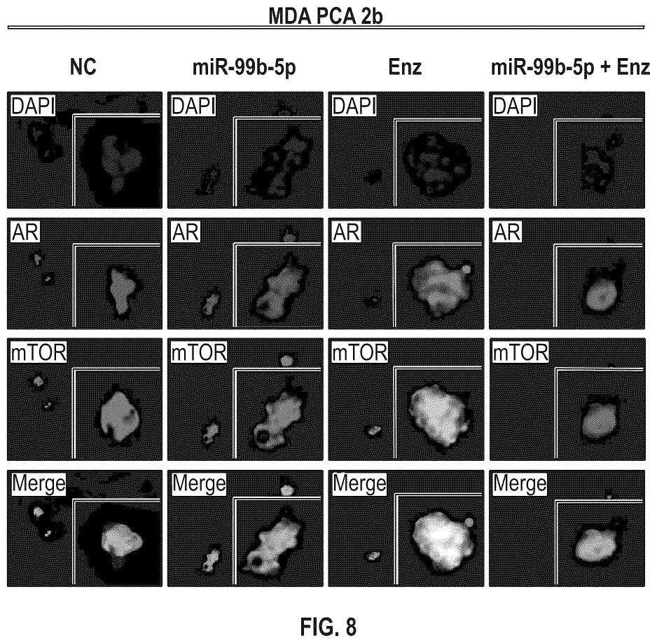

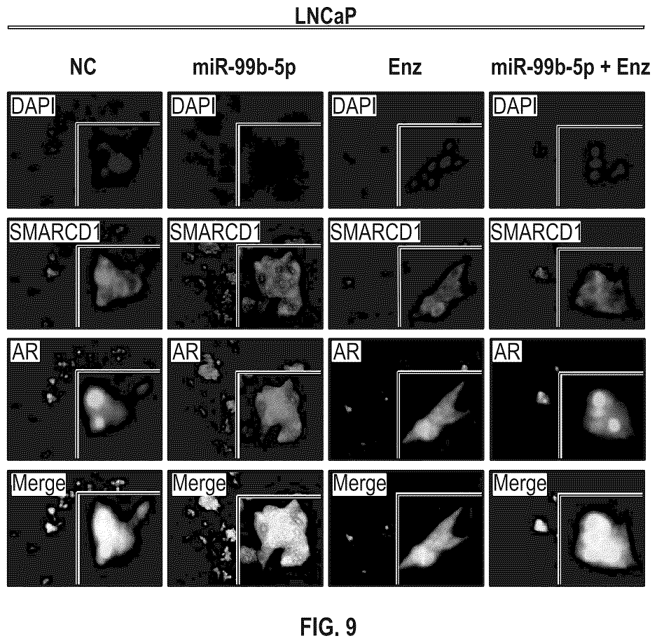

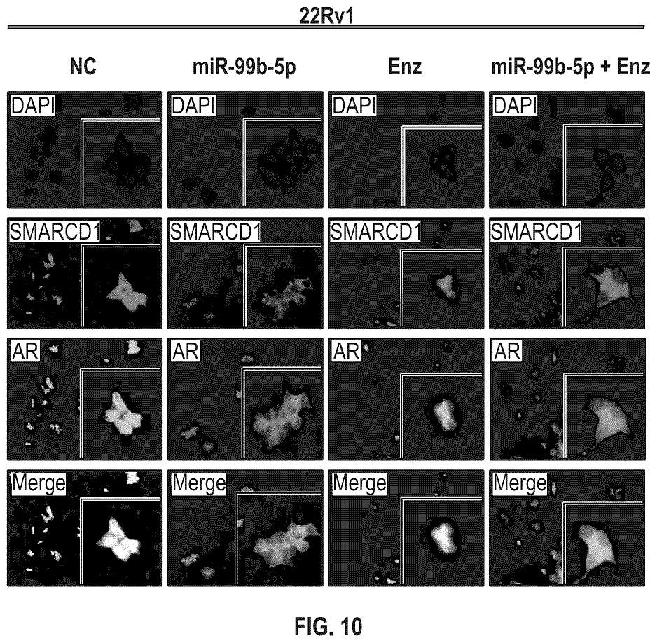

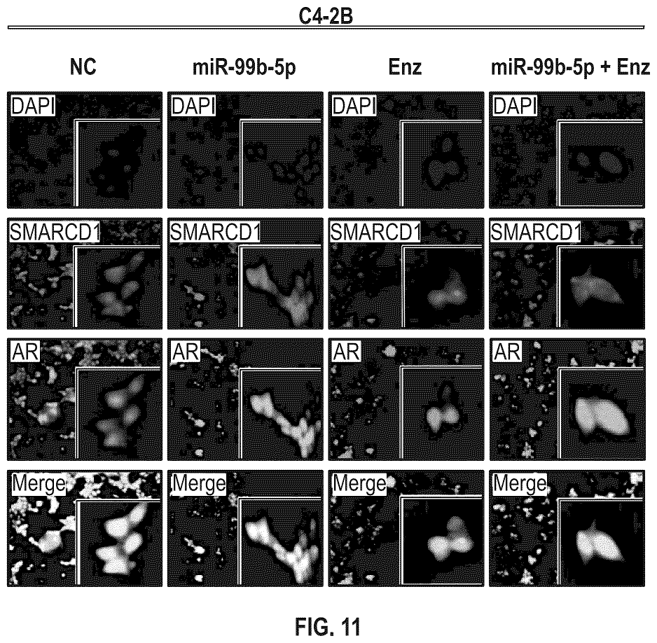

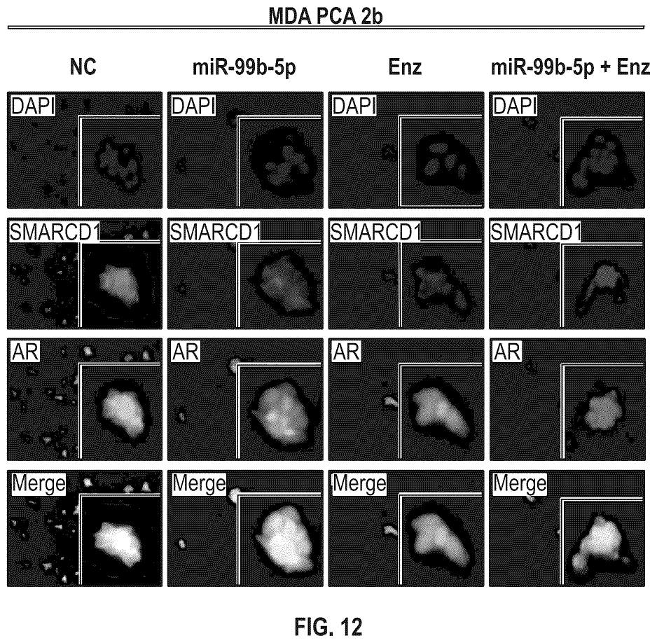

To evaluate the inhibitory effects of miR-99b-5p and Enz on the protein levels and cellular locations of AR/AR-V7, mTOR and SMARCD1, immunofluorescence and western blot assays were performed in the AR-positive lines, including androgen-dependent EA PCa (LNCaP), EA CRPC (22Rv1, C4-2B), and AA PCa (MDA PCa 2b) cells in the absence and/or presence of miR-99b-5p and/or Enz. Specifically, the PCa cell lines were transfected/treated with nonsense/scrambled RNA, miR-99b-5p, 20 mM of Enz, or miR-99b-5p/Enz combination for 48 hr. As shown in FIG. 4A, transfection of miR-99b-5p mimic caused a generalized reduction of mTOR (red fluorescence) and AR (green fluorescence) protein levels, compared to the NC, in all the four PCa cell lines. In contrast, no reduction of mTOR and AR signals was observed in any of the PCa cell lines when treated with Enz. These results reflected the function of Enz as an AR antagonist, which is not regulating the protein level of AR. Intriguingly, almost exclusive cytoplasmic mTOR and AR signals were detected in PCa cells under treatment of either miR-99b-5p, Enz, or miR-99b-5p/Enz combination (FIG. 4A), suggesting both miR-99b-5p and Enz treatments block the nuclear translocation of AR and mTOR. Likewise, transfection of miR-99b-5p mimic decreased the protein levels of SMARCD1 (green fluorescence, FIG. 4B) in LNCaP, 22Rv1, C4-2B and MDA PCa 2b cells. Also, either treatment of miR-99b-5p, Enz, or miR-99b-5p/Enz combination sequestered SMARCD1 in cytoplasm and the nuclear translocation of SMARCD1 was blocked (green fluorescence and merged images, FIG. 4B). Taken together, the results suggested that miR-99b-5p targets/suppresses protein expression of AR, mTOR and SMARCD1, while both miR-99b-5p and Enz inhibit nuclear translocation of AR, mTOR and SMARCD1. Additionally, the full panels of immunofluorescence staining images for AR, mTOR, and SMARCD1 in all PCa cell lines under different treatments were shown in FIGS. 5-12.

Differential Protein Levels of AR/AR-V7, pAR/pAR-v7, mTOR, and SMARCD1 in Cytoplasm and Nuclei of EA and AA PCa Cells in the Presence Absence of miR-99b-5p Mimic and/or Enz

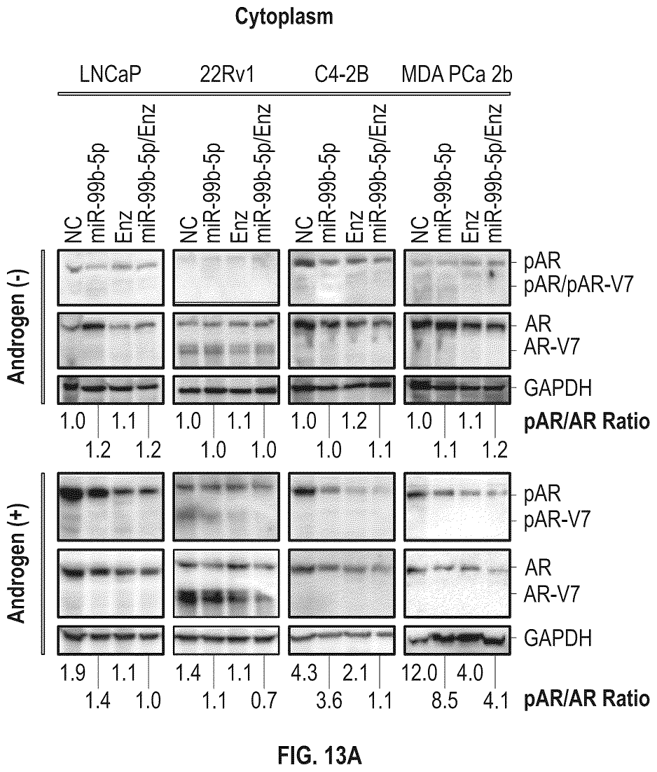

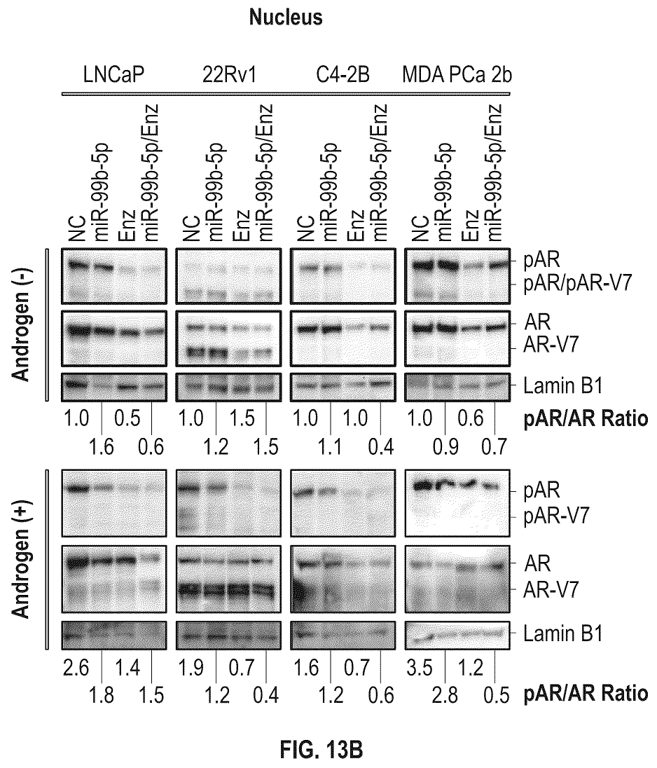

To verify the cellular locations of AR, mTOR, and SMARCD1 under the treatments, western blot analyses were performed using the cytoplasmic and nuclear lysates from EA PCa (LNCaP, 22Rv1 and C4-2B) and AA PCa (MDA PCa 2b) cells grown in the androgen-containing media with the presence/absence of miR-99b-5p and/or Enz. As shown in FIGS. 4C and 4D, AR, mTOR and SMARCD1 levels were decreased in both cytoplasmic and nuclear fractions in LNCaP, C4-2B, and MDA PCa 2b cells treated with miR-99b-5p mimic vs. NC. Whereas, the western blot results revealed that slight to no reduction in cytoplasmic AR and mTOR signals and nuclear mTOR were detected in the 22Rv1 (EA CRPC) cells in response to miR-99b-5p. Notably, remarked decrease in cytoplasmic and nuclear AR/AR-V7, mTOR and SMARCD1 levels were observed when PCa cells were treated with combination of miR-99b-5p and Enz (FIGS. 4C and 4D). Phosphorylation states of cytoplasmic and nuclear AR/AR-V7 were reduced in aIn the presence of 20 mM Enz, phosphorylation states of cytoplasmic/nuclear AR were significantly decreased in the Enz-responding LNCaP, C4-2B and MDA PCa 2b cells, but not in Enz-resistant 22Rv1 cells (particularly pAR level in cytoplasm, in FIG. 4C). Moreover, a synergistic inhibition of pAR was observed in all PCa cell lines (including 22Rv1) under the combined miR-99b-5p/Enz treatment (miR-99b-5p+Enz groups, in FIG. 4C). These results strongly suggested that miR-99b-5p and Enz can effectively inhibit EA and AA PCa, and miR-99b-5p mimic can further sensitize the CRCP cell line 22Rv1 to Enz. Taken together, miR-99b-5p/Enz combined treatment could potentially serve as a novel therapy for Enz-resistant CRCP and androgen-independent AA PCa. Note that the phosphorylation states of cytoplasmic/nuclear AR were shown as basal levels in all treatment groups across four cell lines in the absence of androgen (pAR/AR ratios in FIGS. 13A and 13B, top panels). In the presence of androgens, cytoplasmic/nuclear AR was highly phosphorylated in NC treated cells. However, pAR states were reduced under treatment of miR-99b-5p, Enz, or miR-99b-5p/Enzl1 the PCa cells treated with miR-99b-5p.

In the presence of 20 mM Enz, phosphorylation states of cytoplasmic/nuclear AR were significantly decreased in the Enz-responding LNCaP, C4-2B and MDA PCA 2b cells, but not in Enz-resistant 22Rv1 cells (particularly pAR level in cytoplasm, in FIG. 4C). Moreover, a synergistic inhibition of pAR was observed in all PCa cell lines (including 22Rv1) under the combined miR-99b-5p/Enz treatment (miR-99b-5p+Enz groups, in FIG. 4C). These results strongly suggested that miR-99b-5p and Enz can effectively inhibit EA and AA PCa, and miR-99b-5p mimic can further sensitize the CRCP cell line 22Rv1 to Enz. Taken together, miR-99b-5p/Enz combined treatment could potentially serve as a novel therapy for Enz-resistant CRCP and androgen-independent AA PCa. Note that the phosphorylation states of cytoplasmic/nuclear AR were shown as basal levels in all treatment groups across four cell lines in the absence of androgen (pAR/AR ratios in FIGS. 13A and 13B, top panels). In the presence of androgens, cytoplasmic/nuclear AR was highly phosphorylated in NC treated cells. However, pAR states were reduced under treatment of miR-99b-5p, Enz, or miR-99b-5p/Enz combination (pAR/AR ratios in FIGS. 13A and 13B, bottom panels).

Overexpression of miR-99b-5p Induces Cell Apoptosis and Diminishes Cell Viability in PCa Cell Lines and Organoid Model

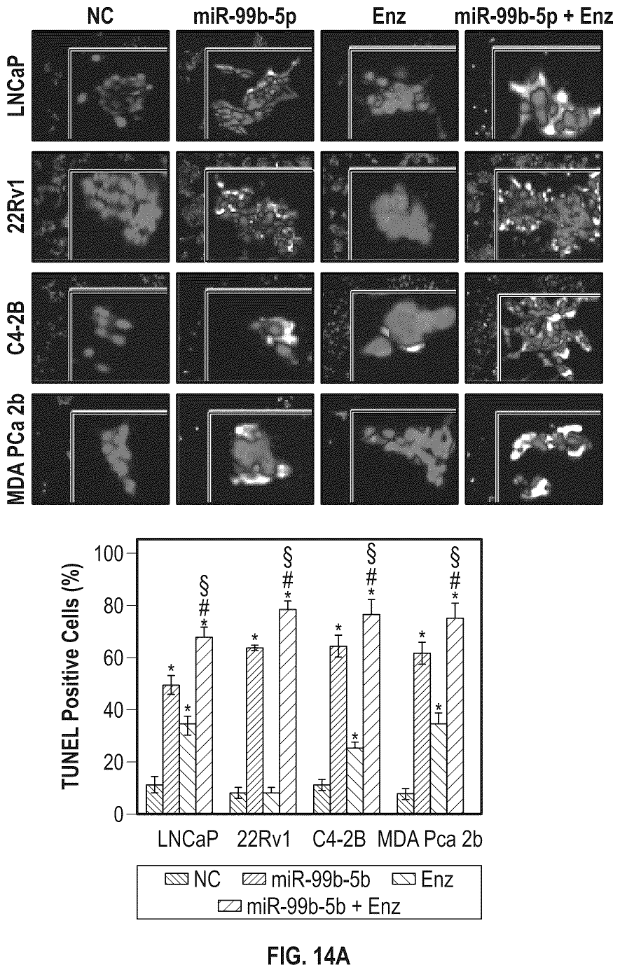

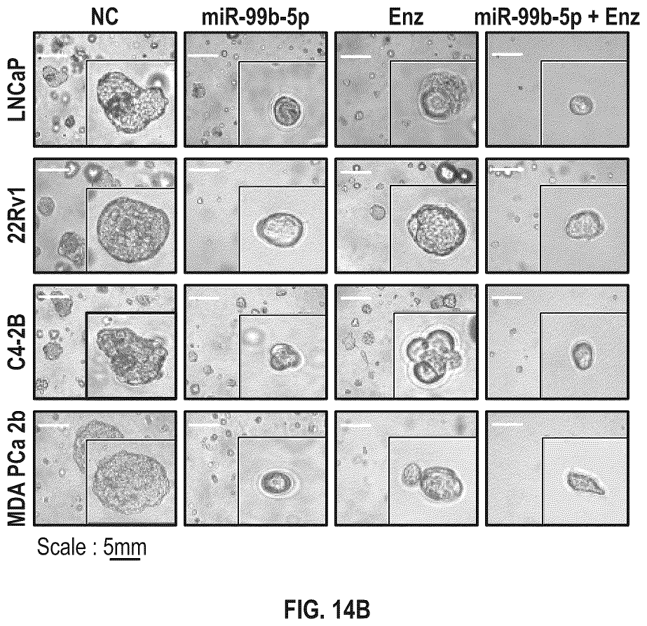

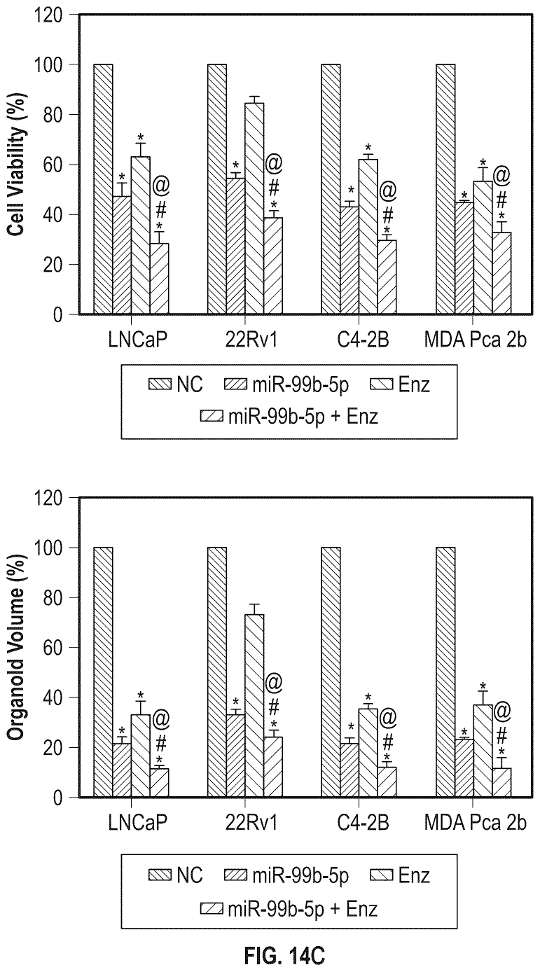

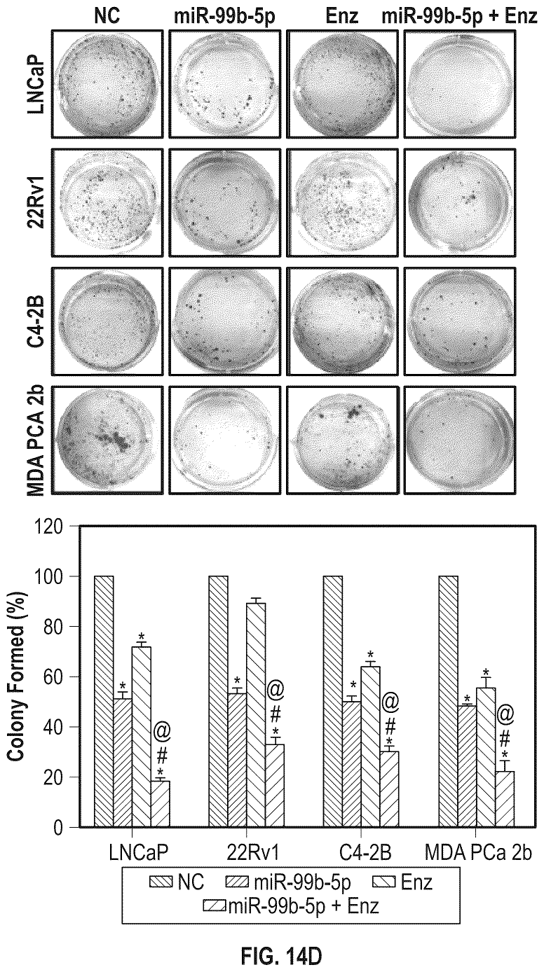

To further assess the inhibitory effects of miR-99b-5p and/or Enz treatments in EA CRCP and AA PCa cells, in-vitro functional assays (TUNEL, MTT, colony forming assays) were conducted in 2D (monolayer) or 3D (organoid) PCa cultures treated with NC, miR-99b-5p, Enz, and miR-99b-5p/Enz combination. As shown in FIG. 14A, TUNEL assay results have demonstrated enhanced DNA breakages (i.e., increased red fluorescent signals in nuclei) in the miR-99b-5p mimic vs. NC transfected in LNCaP, 22Rv1, C4-2B and MDA PCa 2b. In addition, Enz treatments significantly enhanced DNA breakage/cell apoptosis (i.e. increased red fluorescent signals in nuclei, accounting for 50-65% of total cells) in all EA and AA PCa cells, except the resistant 22Rv1 cells (FIG. 14A). Notably, a drastically synergistic effect of apoptotic induction was observed across all the EA and AA PCa cell lines, including the Enz-resistant 22Rv1 (i.e. evident from the 70-80% of TUNEL-positive cells in all PCa cell lines under combination treatment, FIG. 14A).

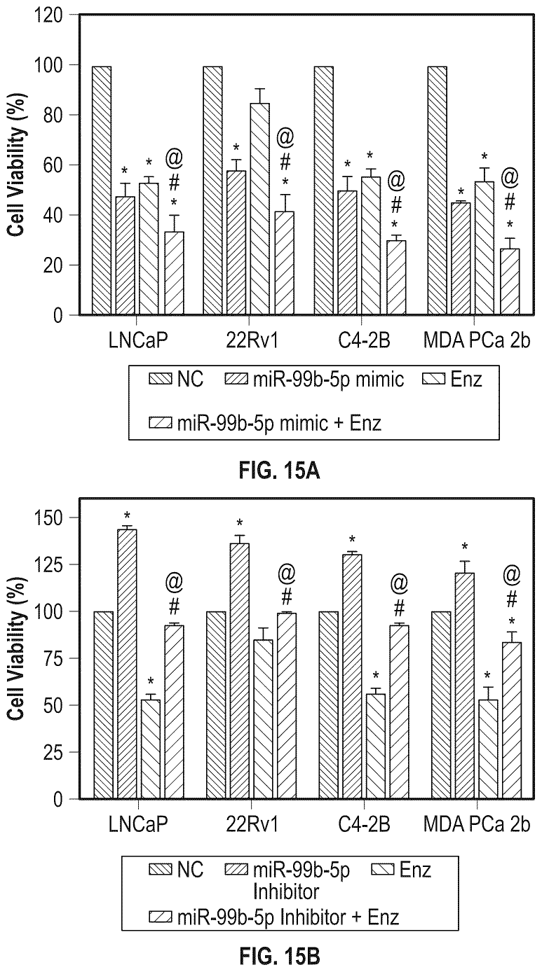

The cell viabilities were assessed by MTT assays in the organoid cultures developed from LNCaP, 22Rv1, C4-2B, and MDA PCa 2b under treatments of NC, miR-99b-5p mimic, Enz and/or miR-99b-5p/Enz combination. Specifically, transfections of miR-99b-5p mimic resulted in significant reductions (i.e. 40-55% decrease) of cell viabilities in all PCa organoids. On the other hand, the Enz treatments resulted in moderate reduction (40-45% decrease) of cell viabilities in LNCaP, C4-2B and MDA PCa 2b cells, while a slight decrease (~10% reduction) in cell viability was observed in 22Rv1 organoid culture (FIG. 14C, top panel). The miR-99b-5p/Enz combination, again, demonstrated a synergistic effect on inhibiting cell growth (ranging from 60-70% decrease in cell viabilities) in all the PCa organoids, including the Enz-resistant 22Rv1 organoid (miR-99b-5p+Enz groups, FIG. 14C, top panel). In contrast, transfection of miR-99b-5p inhibitor resulted in increased cell viabilities in all EA and AA PCa cell lines (miR-99b-5p inhibitor vs. NC, and miR-99b-5p inhibitor/Enz vs. Enz, FIGS. 15A-15B). These results, again, have confirmed the synergistic inhibitory effect of cell growth by treating the EA CRPC and AA PCa with miR-99b-5p mimic/Enz combination therapy. Consistent with the cell viability results, a significant reduction in organoid volumes was observed in all the EA and AA PCa treated with miR-99b-5p or Enz. Furthermore, a significantly synergistic reduction of organoid volumes was observed in response to miR-99b-5p/Enz combined therapy in all EA and AA PCa organoids (FIG. 14C, bottom panel).

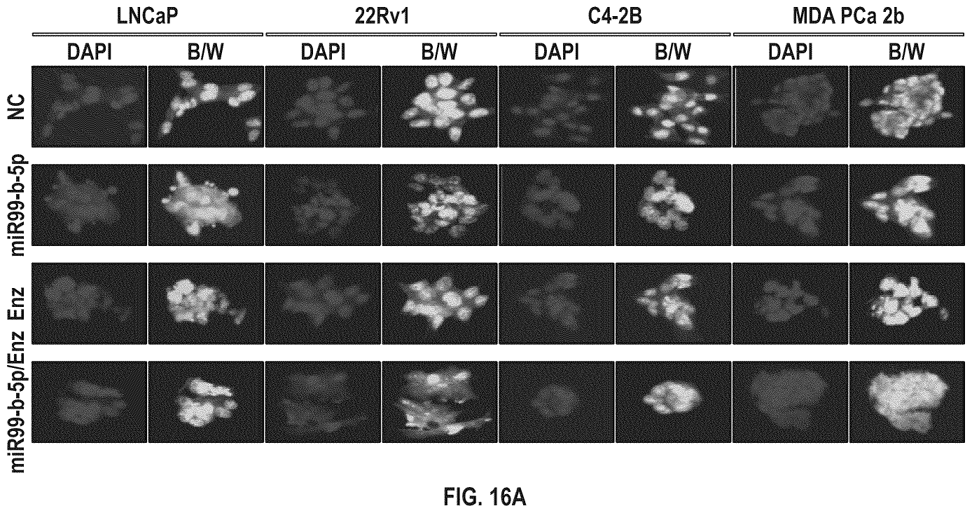

Next, the clonogenic (colony forming) assays were employed to examine the inhibitory effects of miR-99b-5p and/or Enz on LNCaP, 22Rv1, C4-2B, and MDA PCa 2b. Compared to the negative control (nonsense RNA/vehicle control), transfection of miR-99b-5p mimic significantly decreased the cell densities in all the EA and AA PCa cell lines. On the other hand, a significant suppression of cell growths (with 35-50% reduction of cell densities) was observed in Enz-responding LNCaP, C4-2B and MDA PCa 2b treated with Enz vs. NC. As anticipated, 22Rv1 has demonstrated a resistance to Enz (with only 10% decrease in cell density compared to NC, FIG. 14D). The combination of miR-99b-5p (inhibiting AR/AR-V7, mTOR and SMARCD1 levels and their nuclear translocations) and Enz (inhibiting AR/AR-V7 activation) created synergistic effects on suppressing colony forming (with 70-85% decrease in cell densities) in all EA and AA PCa cell lines, including Enz-resistant 22Rv1 (FIG. 14D). Morphological changes in cell shapes and nuclei were observed in EA and AA PCa cells treated with miR-99b-5p, Enz, or miR-99b-5p/Enz (FIGS. 16A-16B), indicating that the treatments might cause cell damage/injury and induce cell apoptosis.

In summary, all the TUNEL, MTT and clonogenic assays have shown consistent results: 1) miR-99b-5p enhances cell apoptosis and inhibits cell viability/growth; 2) Enz effectively induces cell apoptosis and suppresses cell viability/survival in EA and AA PCa, except the Enz-resistant CRPC); 3) miR-99b-5p/Enz combination drastically enhances the cytotoxicity and inhibits cell growth in EA PCa, Enz-sensitive or resistant CRPC, and AA PCa. These results, again, strongly implicate a synergistic inhibitory effect is induced when combining miR-99b-5p with Enz for PCa treatment. Particularly, miR-99b-5p sensitizes CRPC and AA PCa to AR antagonist such as Enz.

mTOR ChIP-qPCR Assays Revealed that miR-99b-5p Mimic and Enz Modulate the Recruitment of AR/mTOR Complex to its Target Genes

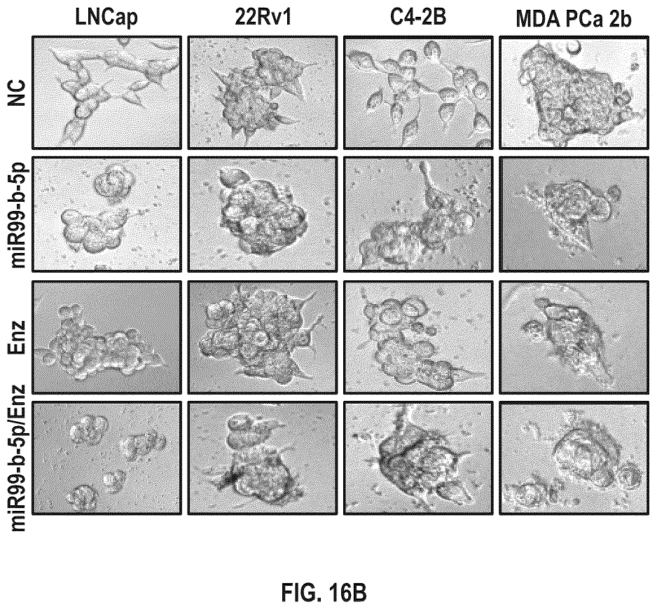

mTOR forms complex with AR and the nuclear mTOR regulates the metabolic gene transcription/reprogramming in CRPC. It was hypothesized that miR-99b-5 (that negatively regulates mTOR and AR expression and nuclear translocation) plays a crucial role in regulating metabolic reprogramming in CRPC. Therefore, overexpression of miR-99b-5p theoretically disrupts the recruitment of nuclear mTOR and nuclear mTOR/AR complex onto their target genes, consequently inhibiting the expression of metabolic genes in CRPC. To validate this hypothesis, mTOR ChIP assays were performed to immunoprecipitate the mTOR/DNA and mTOR-AR/DNA complexes from LNCaP, 22Rv1, C4-2B and MDA PCa 2b cells treated with NC, miR-99b-5p mimic, Enz, or miR-99b-5p/Enz combination. After mTOR ChIP assays, qPCR assays were conducted to examine the expression levels of the mTOR target genes including KLK3, ENO1, SLC26A3 and TMPRSS2. Specifically, KLK3 encodes PSA, ENO1 and SLC26A3 are metabolic genes actively involved in CRPC, and TMPRSS2 is an androgen-driven gene promoting PCa progression/metastasis. ENO1 and TMPRSS2 were identified as mTOR target genes, and KLK3 and SLC26A3 were identified as mTOR/AR co-target genes. As anticipated, the mTOR ChIP-qPCR assays have revealed that mTOR (or mTOR/AR) was enriched on KLK3, ENO1, SLC26A3 and TMPRSS2 genes in LNCaP, 22Rv1, C4-2B, and MDA PCa 2b (NC treatment, FIGS. 17A and 17B). In contrast, miR-99b-5p mimic significantly reduced the mTOR occupancies on these four genes in all cell lines, including EA PCa, CRPC and AA PCa cell lines (miR-99b-5p vs. NC, FIGS. 17A and 17B). Whereas, Enz treatments significantly reduced the recruitment of mTOR (or mTOR/AR) to KLK3, ENO1, SLC26A3, and TIPRSS2 in LNCaP, C4-2B, and MDA PCa 2b, but not to KLK3 and SLC26A3 in the Enz-resistant 22Rv1 (miR-99b-5p/Enz vs. NC, FIGS. 17A and 17B). Drastically, the combination of miR-99b-5p and Enz exerted a synergistic effect to further decrease the occupancies of mTOR (or mTOR/AR) on these genes in all the tested PCa cell lines, including the Enz-resistant CRPC and AA PCa (miR-99b-5p/Enz vs. miR-99b-5p and miR-99b-5p/Enz vs. Enz, FIGS. 17A and 17B). Taken together, these results suggested that miR-99b-5p and Enz effectively block the recruitment of mTOR (or mTOR/AR) to the target genes involved in the metabolic reprogramming in CRPC and/or PCa progression to CRPC. Furthermore, the combination of miR-99b-5p and Enz triggers a synergistic effect to significantly block the recruitment of mTOR and mTOR/AR onto their target genes, potentially serving as a novel therapeutic strategy for treating the resistant CRPC and aggressive AA PCa.

Discussion for a Combination Therapy of miR-99b-5p and Androgen Receptor Antagonists for Treating Castration-Resistant Prostate Cancer

It has been evident that therapeutics mediating AR inhibition (using AR antagonist, such as Enz) are primarily effective but ultimately developing resistance to ADT development, which is an event termed CRPC. This development/progression to CRPC typically leads to an incurable disease. Therefore, a validation of important biomarkers is a crucial step for detecting and observing the progression of aggressive PCa to CRPC. There is an urgent need for developing both protein and gene-based biomarkers, and microRNAs are well considered as potential diagnostic and/or prognostic biomarkers (in tissue, blood, serum/plasma, and urine of PCa patients) for PCa progression to CRPC. There is potential in developing reciprocal miR-99b-5p/nuclear mTOR (downregulated/upregulated) as a prognostic biomarker for aggressive AA PCa and other metastatic solid tumors. Furthermore, miR-99b-5p/mTOR/AR signaling axis may play crucial roles in promoting AA PCa aggressiveness and also involved in the development of CRPC. Additionally, miR-99b-5p overexpression drastically sensitized the aggressive AA PCa to docetaxel. The functional roles of miR-99b-5p in modulating mTOR/AR/SMARCD1 signaling axis in AA PCa aggressiveness and CRPC progression were further explored. According to TargetScan algorithm, miR-99b-5p is predicted to target/regulate ~60 genes, including mTOR and SMARCD1. AR was recently confirmed as a direct target of miR-99b-5p by previous studies. Also, nuclear AR and mTOR are transcriptional regulators for hundreds of genes in AA PCa and CRCP. Taken together, miR-99b-5p potentially functions as a critical epigenomic driver for AA PCa aggressiveness and CRPC development/progression.

Enz has frequently been used as a first line therapeutic compound for CRPC. However, the drug resistance mechanisms underlying the advanced PCa (such as CRPC and aggressive AA PCa) still remains elusive. The Enz resistance observed in some CRPCs is considerably thought to occur via constant activation of AR, bypass of the AR mechanism, and development of AR independence. A panel of PCa cell lines, including LNCaP (androgen-responsive metastatic EA PCa), C4-2B (CRPC developed from parental LNCaP) and 22Rv1 (Enz-resistant CRPC), and MDA PCa 2b (androgen-independent AA PCa) were used to study the functional impacts of miR-99b-5p/mTOR/AR/SMARCD1 signaling axis in PCa aggressiveness and drug resistance in EA and AA PCa. Previously, it was identified that AR signaling pathway is upregulated in AA PCa vs. EA PCa, and a set of AR-target genes are overexpressed in AA PCa vs. EA PCa. The integrative genomic analysis further identified miR-99b-5p/mTOR (down-/up-regulated) as a core miRNA-mRNA reciprocal pairing contributing to the upregulation of mTOR and VEGF signaling in AA PCa. Particularly, nuclear mTOR (and pmTOR) is enriched in AA PCa vs. EA PCa, and transfection of miR-99b-5p inhibits mTOR/AR expression and blocks the nuclear translocation of mTOR and AR. The AR-coactivator, SMARCD1, is critical for activating AR-target genes in nucleus for CRPC progression.

In the present disclosure, the functional roles of miR-99b-5p in modulating mTOR/AR/SMARCD1 signaling in MDA PCa 2b (an AA PCa cell model with similar features of CRPC C4-2B were explored, that is androgen-independent but responds to Enz treatment) vs. LNCaP (EA PCa, androgen-dependent and Enz-sensitive PCa) and 22Rv1 (androgen-independent and Enz-resistant PCa). The enrichment of nuclear mTOR, AR and SMARCD1 in MDA PCa 2b has indicated a more aggressive tumor phenotype in AA PCa vs. EA PCa (i.e., LNCaP, an androgen-dependent EA PCa with much lower nuclear mTOR, AR, and SMARCD1). On the other hand, the enrichment of nuclear AR/AR-V7 and SMARCD1 in C4-2B and 22Rv1 (demonstrating similar aggressiveness to MDA PCa 2b) reflects the importance of AR/SMARCD1-signaling in CRPC (FIGS. 2A-2B). In either case, miR-99b-5p mimic inhibits tumor growth/viability and sensitizes Enz-induced cytotoxicity in AA PCa and EA CRPC (FIGS. 14A-14D). Further mapping of global mTOR and AR occupancies across genomes (i.e., using RNA-seq) of MDA PCa 2b vs. 22Rv1 and C4-2B may further identify the similarity/difference in metabolic rewiring and mTOR/AR/SMARCD1-mediated signaling in AA PCa vs. EA CRPC.

It has been suggested that the upregulation of the AKT/mTOR signaling pathway plays a key role on activating AR signaling cascade and promoting drug resistance in PCa cells. The constitutive activation of AR via mTOR upregulation represents a complex pathway for promoting aggressiveness and drug resistance in CRPC. In addition, the aggressiveness of PCa cells oftentimes demonstrate hyperactivation of mTOR signaling pathway, strongly implicating the involvement of mTOR-mediated signaling in aggressiveness and/or Enz-resistance in refractory CRPC. The exposure to the AR agonist R1881 activates AR and triggers the nuclear translocation of mTOR. Furthermore, the mTOR-dependent metabolic reprogramming has been demonstrated as a critical process for transcriptional regulating the metabolic gene expression profiles required for CRPC, even in the absence of androgens. For the inhibition AR-signaling, metastatic PCa patients are treated with ADT, that significantly extend overall survival of the patients. However, the ADT targeted patients eventually progress to castration-resistance, wherein tumor cells grow and metastasize even at castrated level of androgens.

Earlier findings have shown that CRPC remains constant to AR signaling and such different mechanisms including AR upregulation and activated AR mutations. Based on such mechanism, an alternative AR splice variant has been known to cause reactivation of AR signaling in CRPC. Compared to full-length AR, AR-V7 spliced isoform has previously been shown to differentially regulate the downstream genes. AR-V7 can inhibit a specific set of tumor suppressor genes, thereby causing a castration-resistance phenotype in PCa. Also, AR-V7 variant lacks the ligand binding domain (LBD), and therefore is resistant to the AR antagonists (i.e. abiraterone and enzalutamide), which are the currently available therapy for CRPC. To solve this problem, drugs/compounds that causes the degradation of both full-length AR and AR-V7 are currently under development.

miR-99b-5p serves as a therapeutic molecule to simultaneously inhibit AR and AR-V7 in the PCa, evident from the significant reduction of AR in all cell lines and AR-V7 in 22Rv1 treated with miR-99b-5p or miR-99b-5p/Enz (FIGS. 4A-4C).

miR-99b-5p also targets and inhibits SMARCD1, a coactivator of AR. SMARCD1 (BAF60a) has been known as a member of SWI/SNF family of proteins and it is well recognized to interact with the Ligand Binding Domain (LBD) of AR through its FXXFF motif in an androgen-dependent manner. In addition, it has been shown to bind with glucocorticoid receptor (GR) and needs the docking site for the chromatin remodeling BRG1 complex. SMARCD1 has been reported to be regulated by hepatocyte-specific miRNA miR-122, and has been validated to also be targeted/inhibited by miR-99b-5p in the present disclosure (FIGS. 4A-4C). Consistent with the earlier report, it is shown and validated that miR-99b-5p negatively regulates SMARCD1 expression at the protein level (FIGS. 4A-4C).

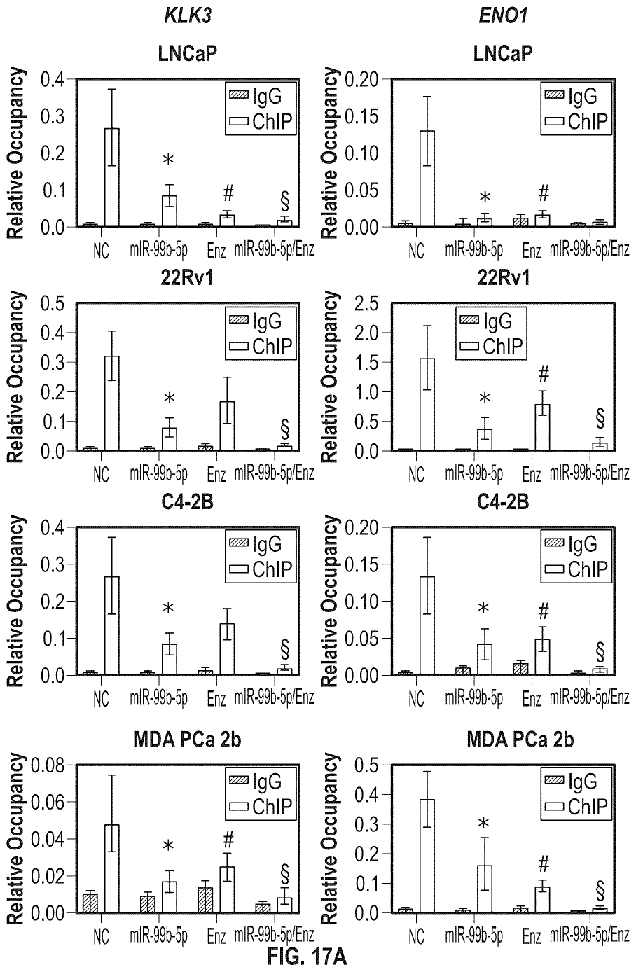

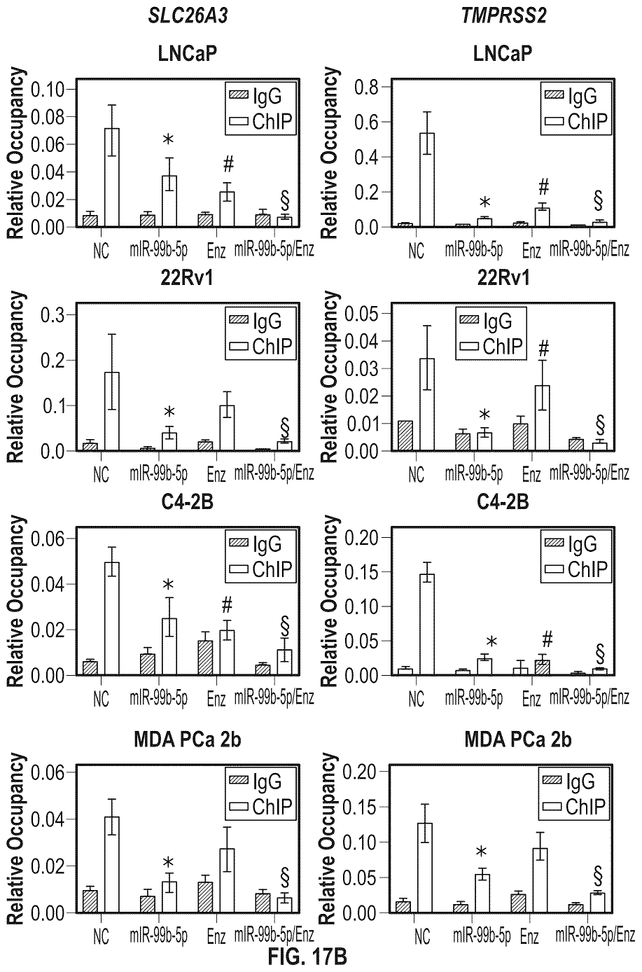

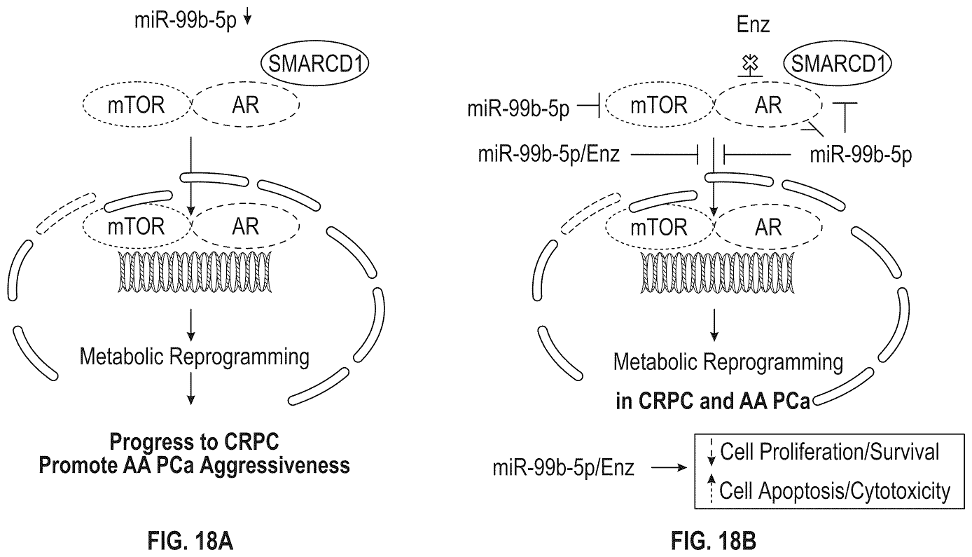

Downregulation or deletion of miR-99b-5p could cause upregulation of mTOR, AR and AR coactivator SMARCD1, consequently activate a mTOR/AR-mediated metabolic gene reprogramming. This metabolic gene reprogram could further lead to the PCa aggressiveness and/or progression to CRPC disease. Taken together, the miR-99b-5p/mTOR/AR/SMARCD1 signaling axis may play important functional roles in promoting AA PCa aggressiveness and CRPC progression (as shown in FIG. 18A). On the other hand, overexpression of miR-99b-5p (i.e. via transfection of miR-99b-5p mimic) targets/inhibits AR, mTOR and SMARCD1 simultaneously and blocks the translocation of mTOR/AR/SMARCD1 complex from cytoplasm to nucleus, consequently suppressing cell proliferation/survival and enhancing the cell apoptosis in PCa (especially AA PCA and CRPC). Furthermore, miR-99b-5p overexpression results in suppressing nuclear translocation of mTOR/AR/SMARCD1, thereby inhibiting mTOR/AR-mediated metabolic reprogramming and significantly sensitizes the CRPC and AA PCa to the Enz (or other AR antagonists) (FIG. 18B). To date, this is the first disclosure that investigates the functional roles of miR-99b-5p in the mTOR/AR/SMACR1 signaling axis in AA PCa and Enz-responding/resistant CRPCs. The synergistic inhibitory capacities of the miR-99b-5p/Enz combination may indicate a novel molecular strategy for targeting/treating the CRPC and aggressive AA PCa.

Materials and Methods for a Combination of miR-99b-5p and Enzalutamide or Abiraterone Synergizes the Suppression of EMT-Mediated Metastasis in Prostate Cancer

### Cell Culture Maintenance and Conditions

The human PCa cell lines LNCaP, 22Rv1, C4-2B, and MDA PCa 2b were used in the current study. LNCaP is an EA PCa cell line derived from lymph nodes. C4-2B is a CRPC cell lines derived from EA PCa, 22Rv1 is a CRPC cell line derived from EA patient, while MDA PCa 2b is an AA PCa cell lines derived from bone metastasis. All the PCa cell lines used in this study were purchased from American Type Culture Collection (ATCC, Manassas, VA, USA). RPMI-1640 (Gibco, Waltham, MA, USA) with 10% fetal bovine serum (FBS, Gibco, Waltham, MA, USA) used as the medium to grow LNCaP and 22Rv1 cells. DMEM (Gibco, Waltham, MA, USA) with 10% FBS was used to grow C4-2B cells; BRFF-HPC1 (Athena ES, Baltimore, MD, USA) with 20% FBS was employed to grow MDA PCa 2b cells.

Human umbilical vascular endothelial cells (HUVECs) were maintained at 37° C. with 5% CO₂ in EBM-2 media, as per the manufacturer's instructions. HUVEC cells, at passages of 2-3, were used for the experiments. Furthermore, HUVECs cultured on Matrigel were exposed to the condition medium (CM). The preparation of the CM is described as follows: 1.5×105 cells/mL of PCa cells were grown in 6-well plates coated with collagen I for proper attachment in each well. After 3-4 days, the culture medium was removed and replaced with fresh media in each well. The PCa cells were then incubated for 2-3 days and, thereafter, the media was collected and centrifuged at 1000×g for 5 min. Finally, the obtained supernatants were saved and defined as the CM.

### MicroRNA Transfection and Drug Treatment Schedule in EA and AA PCa Cell Lines

Subsequently, 3×105 cells of LNCaP, 22Rv1, C4-2B, or MDA PCa 2b cells were seeded and grown in a well of the 6-well plate; then, the PCa cells were grown for 24 h. Thereafter, the PCa cells were transfected with either nonsense RNA or miR-99b-5p mimic (Ambion, Austin, TX, USA). The transfected PCa cells were then grown overnight and treated with vehicle, Enz, or Abi for an additional 48 h. The treatment groups were as follows: NC (transfection of nonsense control, and vehicle treatment), miR-99b-5p (transfection of miR-99b-5p mimic, and vehicle), Enz (transfection of nonsense RNA, and 20 μM of Enz), Abi (transfection of nonsense RNA and 10 μM Abi), miR-99b-5p/Enz (combination of miR-99b-5p mimic and 20 μM of Enz), and miR-99b-5p/Abi (combination of miR-99b-5p mimic and 10 μM of Abi). All the cells were grown in 5% CO₂ incubator at 37° C. A total of 20 μM of Enz and 10 μM of Abi were the concentrations used in the experiments, according to previous studies.

### Immunofluorescence Staining for PCa Cell Models

In brief, 4×10⁴ cells were grown on a cover slip for 24 h in 5% CO₂ incubator at 37° C. Thereafter, cells were washed with 1×PBS, fixed in 4% paraformaldehyde, and permeabilized with 0.1% Triton X-100. The fixed and permeabilized PCa cells were blocked for 1 h with 2% BSA in 1×PBS. After blocking, primary antibody was applied to label the protein of interest. After incubating the sample with primary antibody overnight at 4° C., the cells were washed twice with 1×PBS then incubated with secondly antibody conjugated with fluorescence for 1 h at room temperature. Finally, the cells were mounted with DAPI-containing mounting medium (Invitrogen, Waltham, MA, USA). The antibodies used in the study are listed as follows: N-cadherin (catalog #13116, 1:200 dilution, Cell Signaling Technology, Danvers, MA, USA; or catalog #sc-59987, 1:100 dilution, Santa Cruz Biotechnology, Dallas, TX, USA), E-cadherin (catalog #3195, 1:200 dilution, Cell Signaling Technology, Danvers, MA, USA; or catalog #sc-8426, 1:100 dilution, Santa Cruz Biotechnology, Dallas, TX, USA), Vimentin (catalog #5741, Cell signaling, 1:200 dilution, Danvers, MA, USA), Snail (catalog #3879, 1:200 dilution, Cell Signaling, 1:100 dilution, Danvers, MA, USA; or catalog #sc-271977, Santa Cruz Biotechnology, Dallas, TX, USA), and Alexa-Fluor-488-conjugated anti-rabbit and Alexa-Fluor-594-conjugated anti-mouse antibodies (catalog #A32731 and #A32744, 1:500 dilution, respectively, from Invitrogen, Waltham, MA, USA). The immunofluorescence-stained cells were visualized and the images were captured using Olympus fluorescence microscopy (Waltham, MA, USA). The images of the immunofluorescence signals were captured from 3-4 random areas, and image analysis was performed using CellSens V1.18 software (Olympus, Waltham, MA, USA).

### Western Blot Analysis

Western blot assays were performed using the protocol as previously described in Ha et al., “Molecular Insight into Drug Resistance Mechanism Conferred by Aberrant PIK3CD Splice Variant in African American Prostate Cancer”, Cancers 2023, 15, 1337; which is hereby incorporated by reference in its entirety herein. The PCa cells were collected, and total proteins were extracted using M-PER with a pro-tease and phosphatase inhibitor cocktail (Thermo Fisher Scientific, Waltham, MA, USA). Quantification of total protein concentrations were performed using the BCA assay kit (Thermo Fisher Scientific, Waltham, MA, USA). The protein samples were loaded in Bolt 4-12% Bis-Tris mini protein gels (Thermo Fisher Scientific, Waltham, MA, USA) for electrophoresis. The primary antibodies used in the study were as follows: antibodies for E-cadherin (catalog #3195, 1:1000 dilution), N-cadherin (catalog #13116, 1:1000 dilution), Vimentin (catalog #5741, 1:1000 dilution), Snail-1 (catalog #3879, 1:1000 dilution), and 3-actin (catalog #4970S, 1:2000 dilution) from Cell Signaling Technology (Danvers, MA, USA). The secondary antibodies were anti-rabbit IgG-HRP (catalog #4030-05, 1:10000 dilution) and anti-mouse IgG-HRP (catalog #1033-05, 1:5000 dilution) antibodies from Thermo Fisher Scientific (Waltham, MA, USA).

### Evaluation of Tumor Migration by Using Wound Healing Assay

PCa cells were cultured until 95-100% confluence in a 6-well plate. An elongated scratch was created using a pipette tip; the floating cells were then removed and the remaining attached cells were washed with 1×PBS. The cells then underwent treatments of NC, miR-99b-5p mimic, Enz, Abi, miR-99b-5p/Enz, and miR-99b-5p/Abi, as described above. To visualize the migration capacities of the PCa cells with different treatments, cell migrations of all groups were observed and photographed using an inverted phase contrast microscopic system (Olympus, Waltham, MA, USA) at 0 and 48 h. The migration capacities were measured by calculating the migrating distance of the cells between 0 and 48 h. The migrating distance (scratch width at 0 h-scratch width at 48 h) of the NC-treated cells was defined as 100%. Scratch widths at 0 and 48 h were defined as the distances between the two yellow dashed lines at 0 h and 48 h, respectively. The data were generated from 3 independent scratch wound healing assays per treatment group. The statistics and bar graphs were performed and plotted using Prism 9 program (GraphPad Software, La Jolla, CA, USA).

### Transwell Migration Assay

A migration assay was performed in 12-well transwells with a pore size of 4.0 μM (Corning, Wilkes Barre, PA, USA). The PCa cells were first grown for 24 h and then the cells were transfected with either nonsense RNA or miR-99b-5p mimic. Second, 100 μL of transfected PCa cells (1×104) suspended in serum-reduced medium were transferred to the upper chambers of the transwell compartment. A total of 600 μL of medium containing 10% FBS as a chemoattractant was added to the lower chamber of the transwell compartment. Third, the transferred PCa cells were then treated with either vehicle, enzalutamide, or abiraterone for an additional 48 h and subjected to staining of the migrated cells. Specifically, non-migrated cells (cells remaining on the top side of the membrane in the insert) were wiped off using a cotton swab. The migrated cells (cells migrated to the bottom side of the membrane in the insert) were fixed with 100% methanol (Sigma, St. Louis, MO, USA) and stained with 0.5% crystal violet (Sigma, St. Louis, MO, USA) for counting the number of migrated cells to determine the migration capacities of the PCa cells. The cells that migrated to the bottom surface were visualized and counted in 3-4 randomly chosen areas using an inverted microscope (Olympus, Waltham, MA, USA) at 10× magnification. The data were obtained from 3-4 independent experiments. The migrated cells (%) were determined via normalization of the migrated cells in the experimental group to the migrated cells in the NC group. The data were analyzed using the Prism 9 program (GraphPad Software, La Jolla, CA, USA) for graphing and statistical analysis.

### Cell Adhesion Assay

For this procedure, 1×105 of PCa cells were seeded in a 24-well plate coated with 35 μg/mL of collagen and 10 μg/mL fibronectin. Cells were cultured for 24 h before transfection with nonsense RNA or miR-99b-5p mimic. Then, the transfected PCa cells were further treated with vehicle, Enz, or Abi. After treatment for 48 h, the medium was aspirated and the wells were washed thrice with 1×PBS. Furthermore, the cells were then fixed with 100% chilled methanol for 15 min and stained using 0.5% crystal violet for 20 min. Overstained cells were washed 3-4 times with 1×PBS to remove excess dye. The number of adherent cells (stained by crystal violet) was visualized and counted using an inverted phase-contrast microscopic system (Olympus, Waltham, MA, USA). The number of adherent cells (averaged from n=3-4) in the NC group was defined as 100%, and was used to normalize the adherent cells (%) for other experimental groups. The data analyses were achieved using the Prism 9 program (GraphPad Software, La Jolla, CA, USA) for graphing and statistical analysis.

### Angiogenesis (Tube Formation) Assay

A total of 70 μL of Matrigel was placed into each well of a 96-well plate and allowed to polymerize for 1 h at 37° C. HUVEC cells at a passage of 2-3 were then co-cultured with 100 μL of PCa-derived CM. After 24 h, tubular morphology of HUVECs was observed and images were captured using an inverted phase contrast microscopic system optical microscope (Olympus, Waltham, MA, USA) with a 20× objective lens. All observations were performed 3-4 times, and each experiment was repeated 3-4 times.

### Statistical Analysis

All the data were calculated and are presented as mean±standard deviation (SD). The multiple comparisons were analyzed using analysis of variance (ANOVA) with Tukey's post hoc test. The statistics (i.e., significance based on p-values, etc.) were performed using GraphPad Prism 9.0 (Graph Pad Software, La Jolla, CA, USA).

Introduction for a Combination of miR-99b-5p and Enzalutamide or Abiraterone Synergizes the Suppression of EMT-Mediated Metastasis in Prostate Cancer

In the United States, PCa is the most commonly diagnosed cancer and second leading cause of cancer mortality (estimated with 299,010 new cases and 35,250 deaths in 2024) among American men. Due to the high heterogeneity of PCa, the development of optimal treatment options for patients remains challenging because of the poor response towards therapies and tumor recurrence. Therefore, identification of novel drug targets is in urgent need for further developing individualized therapeutic strategies for PCa patients.

Androgen deprivation therapy (ADT) has been considered as a standard regimen for localized and advanced PCa patients. Despite an initial favorable response to ADT, most of the patients experienced disease recurrence within 18-24 months. Ultimately, the disease progresses to castration-resistant prostate cancer (CRPC), a metastatic PCa with poor prognosis. Previous studies have demonstrated that PCa cells exhibit high plasticity (termed ‘lineage plasticity’), allowing PCa cells to adapt to ADT through cellular rewiring (i.e., via upregulation of AR, PI3K, or GATA2 signaling) and, therefore, develop drug resistance. Second-generation hormone therapeutic agents, such as enzalutamide and abiraterone acetate, have been shown to improve overall survival and quality of life in metastatic CRPC patients. Enzalutamide (Enz) is an androgen receptor (AR) antagonist that demonstrates potent efficacy against metastatic CRPC. Mechanistically, Enz blocks the translocation of the AR from the cytoplasm to the nucleus and prevents the binding of AR with its coregulators and chromatin binding sites, subsequently inhibiting the transcription of AR-downstream genes. Abiraterone acetate (Abi) has been utilized as a first-class, selective inhibitor of cytochrome P450 (CYP) 17, a crucial enzyme responsible for extragonadal and testicular androgen synthesis. Abi along with prednisone has also been shown to improve the survival of and reduce side-effects for metastatic CRPC patients. Although both Enz and Abi demonstrated clinical benefits, the overall survival of Enz/Abi-treated patients is only modestly increased and the majority of responders develop resistance over time. For example, most Enz-treated patients who have had drastic reduction in PSA levels eventually develop resistance, with increasing PSA and/or progression of bone metastasis. To date, AR-V7 synthesis, upregulation of CYP17 and/or alteration in AR signaling axis, expression of glucocorticoid receptor (GR), upregulation of PI3K/AKT/MAPK and/or GATA2 signaling, altered tumor microenvironment, and deregulation of microRNAs (miRNAs) have been considered as possible mechanisms involved in the acquired resistance to Enz and Abi. For instance, AR-V7 is a splice isoform lacking the ligand-binding domain (LBD) of AR. Therefore, an increased level of AR-V7 has been associated with disease progression and treatment failure of Enz and Abi in CRPC. Upregulation AR signaling, due to AR gene amplification; increased stability of AR; or mutations in AR, could also lead to Enz and Abi resistance. Due to the clinical challenges observed in CRPC, novel therapeutics that overcome drug resistance are still required.

MicroRNAs (miRNAs) are small non-coding RNAs that epigenetically regulate cellu-lar processes by regulating gene expression at the post-transcriptional level. MiRNAs are frequently deregulated in cancer development/progression and drug resistance; therefore, miRNAs have been implicated as potential biomarkers and treatment response modulators in various cancers, including PCa. Previously, we identified dozens of reciprocal miRNAs/mRNA pairings and associated miRNA-mRNA regulatory networks in African American (AA) PCa disparities. See e.g., Gujrati et al., “MicroRNA-mRNA Regulatory Network Mediates Activation of mTOR and VEGF Signaling in African American Prostate Cancer”, Int. J. Mol. Sci. 2022, 23, 2926; and Wang et al., “Identification and Functional Validation of Reciprocal microRNA-mRNA Pairings in African American Prostate Cancer Disparities”, Clin. Cancer Res. 2015, 21, 4970-4984; each of which are hereby incorporated by reference in their entireties herein. Among these miRNA-mRNA pairings, the down-regulation of miR-99b-5p and upregulation of MTOR has been implicated as a critical epigenetic event that contributes to tumor aggressiveness in AA PCa and the progression of CRPC. See e.g., Gujrati et al., “Downregulation of miR-99b-5p and Upregulation of Nuclear mTOR Cooperatively Promotes the Tumor Aggressiveness and Drug Resistance in African American Prostate Cancer”, Int. J. Mol. Sci. 2022, 23, 9643; and Waseem et al. “Tumor suppressive miR-99b-5p as an epigenomic regulator mediating mTOR/AR/SMARCD1 signaling axis in aggressive prostate cancer”, Front. Oncol. 2023, 13, 1184186; each of which are hereby incorporated by reference in their entireties herein. Furthermore, our studies have shown that miR-99b-5p simultaneously targets and inhibits the expression of MTOR, AR, and SMARCD1 (encoding SMARCD1, an AR coregulator), consequently blocking the translocation of the mTOR/AR/SMARCD1 complex from the cytoplasm to the nucleus, inhibiting the expression of mTOR/AR target genes, reversing metabolic rewiring, and sensitizing AA PCa and CRPC to Enz.

Epithelial-mesenchymal transition (EMT) has been implicated as a critical step during the development of distance metastases from a primary tumor. Specifically, EMT is regulated by transcription factors such as Snail, Slug, ZEB1/2, and Twist1/2. These EMT-transcriptional factors (EMT-TFs) then suppress the expression of E-cadherin (an epithelial marker) and activate the expression of N-cadherin, Vimentin, and fibronectin (mesenchymal markers). The activation of EMT results in reduced cell-cell adhesion and triggers cell detachment from the primary tumor, leading to tumor invasion and migration/metastasis. PI3K/AKT/mTOR and hypoxia/HIF1α signaling, in collaboration with other signaling pathways, induces the expression of EMT-TF genes (i.e., SNAIL, ZEB1/2, and TWIST1/2) and activates EMT in cancers, including PCa. Additionally, AR signaling has also been implicated in the activation of EMT in PCa. Due to the central/unique role of the reciprocal miR-99b-5p/MTOR pairing (downregulated/upregulated in AA PCa) in coordinating PI3K/AKT/mTOR, HIF1α, and AR signaling, we hypothesized that the miR-99b-5p/mTOR/AR/SMARCD1 signaling axis may play a critical functional role in regulating EMT-mediated metastasis in AA PCa and CRPC. To test this hypothesis, we performed a series of functional and biochemical experiments to examine whether transfection/overexpression of miR-99b-5p mimic could inhibit the expression of EMT-TF (Snail) and mesenchymal markers (N-cadherin and Vimentin) and restore the expression of epithelial marker (E-cadherin), ultimately inhibiting the metastatic capacities of AA PCa and CRPC cells. In addition, miR-99b-5p mimic was used as a single agent and in combination with Enz or Abi to assess the inhibitory effects on EMT-mediated migration and angiogenesis capacities in aggressive AA PCa and CRPC cells.

Results for a Combination of miR-99b-5p and Enzalutamide or Abiraterone Synergizes the Suppression of EMT-Mediated Metastasis in Prostate Cancer
Overexpression of miR-99b-5p Mimic and Enz/Abi Treatment Modulate the Expression Levels of N Cadherin, E-Cadherin, Vimentin, and Snail in EA and AA PCa Cells

Four cell lines that were derived from European American (EA) PCa and AA PCa were used in our study. LNCaP is a EA cell line that was derived from a metastatic lymph node lesion of PCa that was AR positive, exhibiting androgen-sensitive tumor growth. C4-2B was derived from a bone metastasis that was established in nude mice after injecting LNCaP-derived, castration-resistant C4-2 cells. 22Rv1 is an EA PCa cell line derived from a xenograft serially propagated in mice after castration-induced regression and relapse of the parental CWR22 xenograft. MDA PCa 2b is a PCa cell line derived from androgen-refractory bone metastasis of an AA PCa patient. MDa PCa 2b cell line was shown to express similar AR and PSA levels as compared to the clinical samples. In summary, LNCaP was used as an AR-positive, androgen-sensitive EA PCa model. C4-2B and 22Rv1 were served as AR-positive EA CRPC models, while MDA PCa 2b was used as an AR-positive, androgen-independent AA PCa model.

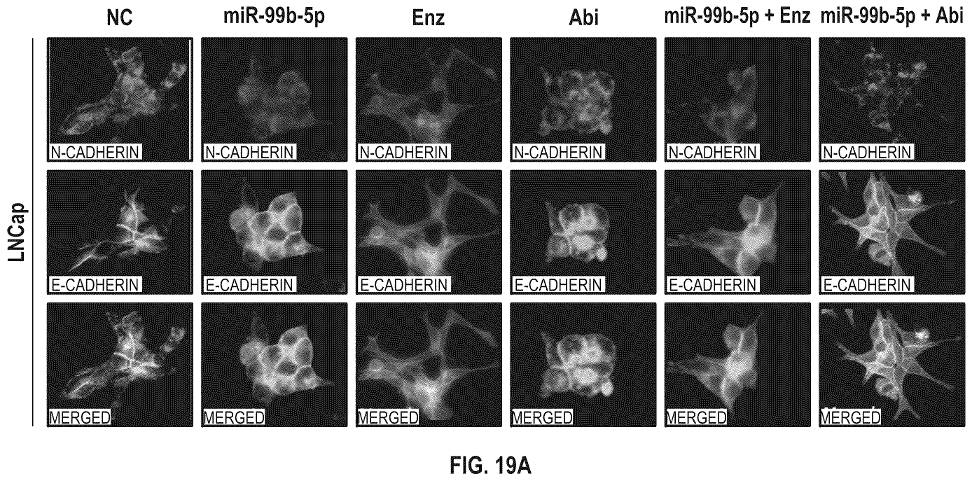

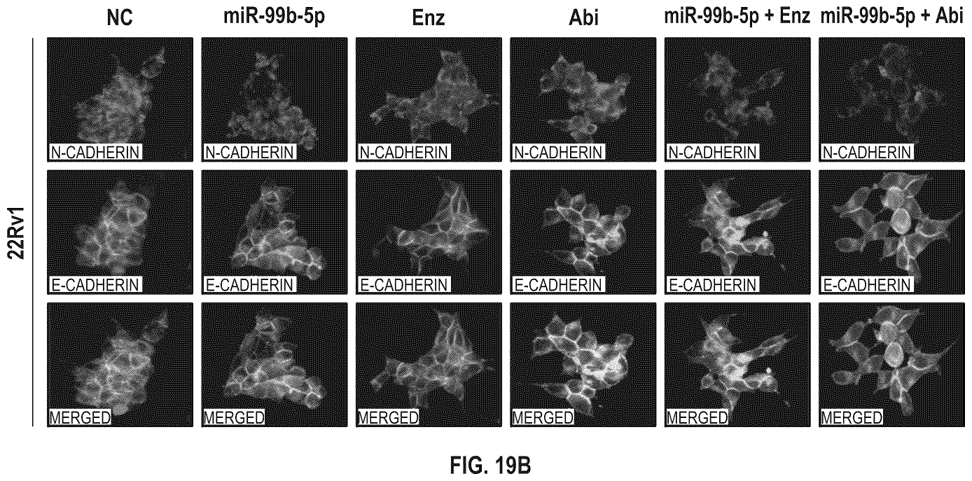

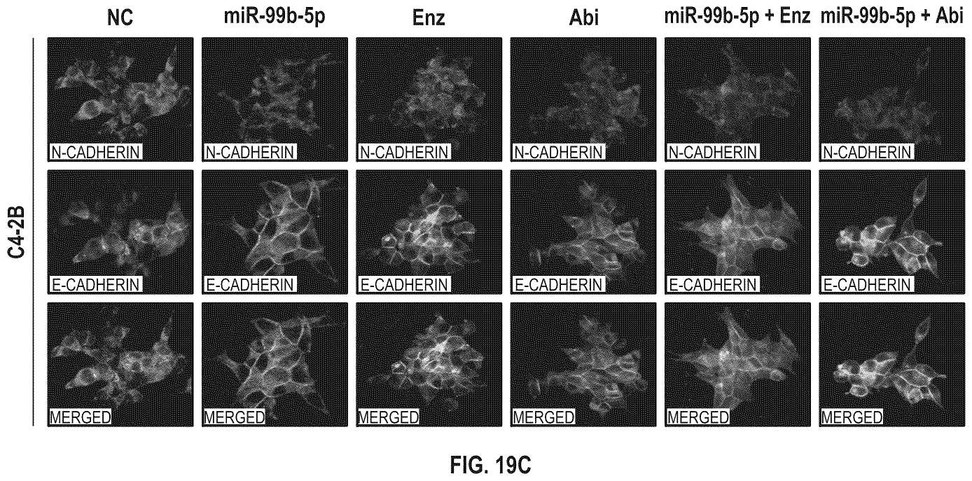

To assess the effects of miR-99b-5p and Enz/Abi on EMT-mediated metastasis, the expression levels of EMT markers E-cadherin (epithelial), Snail (EMT-TF), N-cadherin and Vimentin (mesenchymal), and Snail were examined using immunofluorescence in EA PCa (LNCaP, 22Rv1, and C4-2B) and AA PCa (MDA PCa 2b) cells transfected with nonsense RNA or miR-99b-5p mimic in the absence/presence of Enz or Abi. As shown in FIGS. 19A-19D, overexpression of miR-99b-5p mimic resulted in a generalized reduction in N-cadherin (green fluorescence, top panels) and increase in E-cadherin (red fluorescence, middle panels) protein levels, when comparing miR-99b-5p mimic-treated to NC-treated EA and AA PCa cells. Similarly, either Enz or Abi treatment resulted in the reduction of N-cadherin signals in EA and AA PCa cell lines, when compared to the NC controls (FIGS. 19A-19D). In contrast, an enhanced level of E-cadherin signals was observed in all EA and AA PCa cell lines treated with Enz (or Abi) vs. NC. In addition, a significant reduction in N-cadherin and significant increase in E-cadherin signals were detected in EA and AA PCa cells treated with miR-99b-5p/Enz or miR-99b-5p/Abi combination vs. NC control. Notably, miR-99b-5p/Enz or miR-99b-5p/Abi combination caused lower N-cadherin and higher E-cadherin expression levels compared to miR-99b-5p or Enz/Abi as single agent. Taken together, our results suggested that miR-99b-5p or Enz/Abi effectively inhibit EMT by inhibiting mesenchymal marker N-cadherin and restoring the epithelial marker E-cadherin. Moreover, the combination of miR-99b-5p mimic with Enz or Abi resulted in additive inhibition of EMT, as compared to miR-99b-5p mimic or Enz/Abi alone.

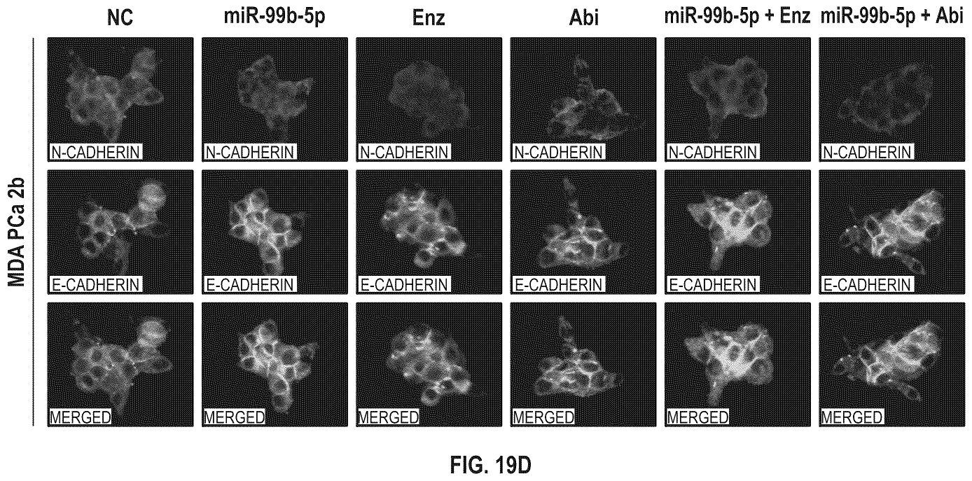

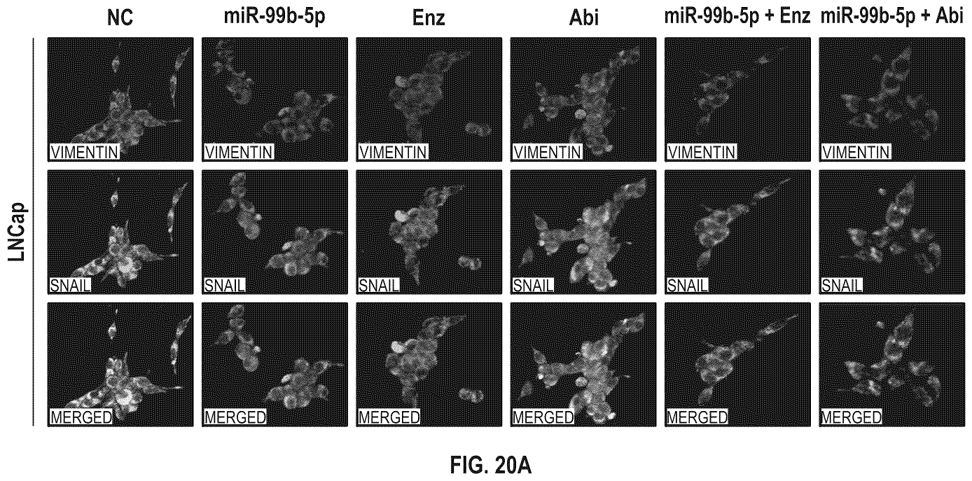

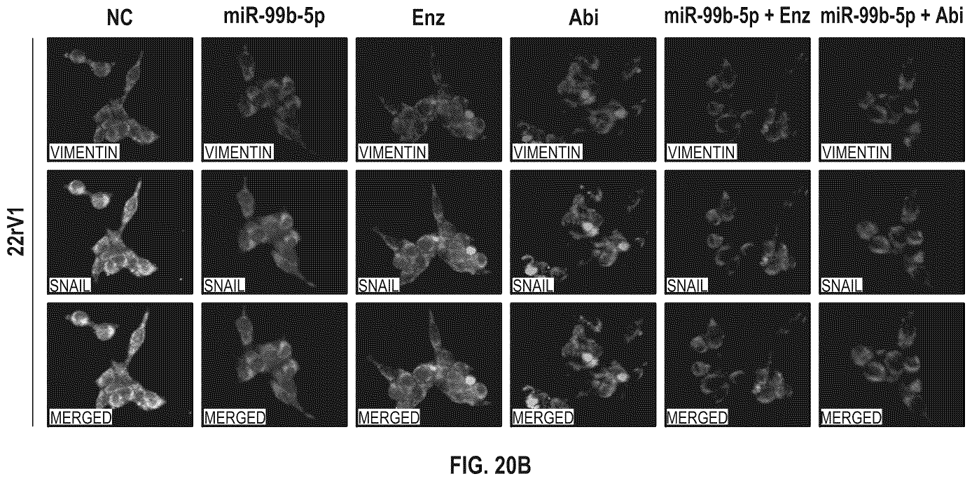

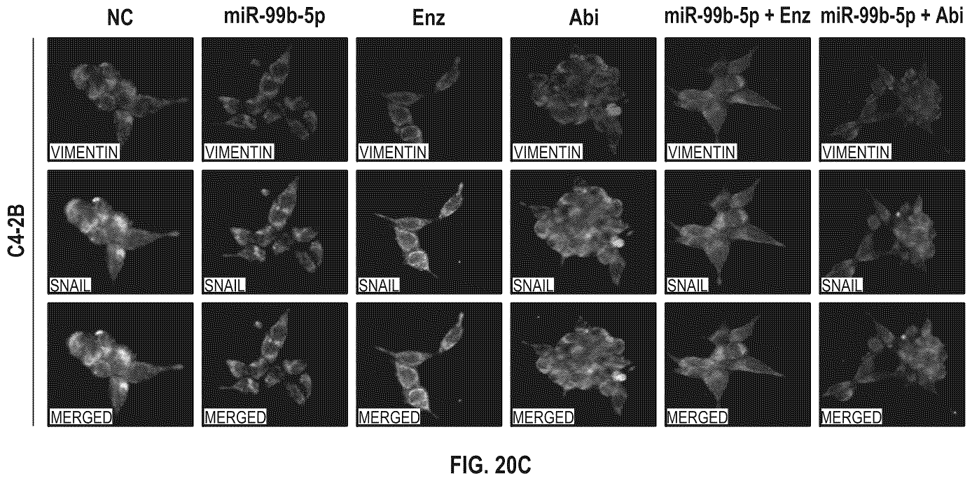

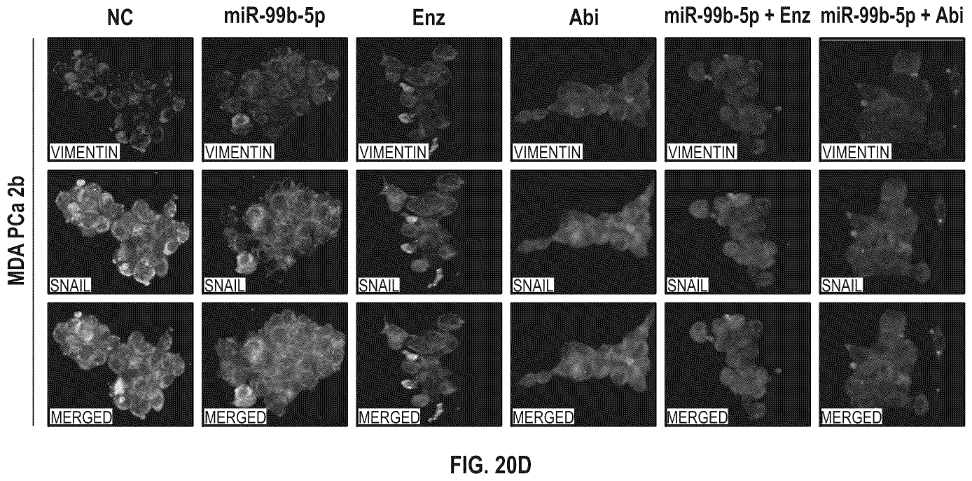

As shown FIGS. 20A-20C, overexpression of miR-99b-5p mimic caused a reduction in both Vimentin (green fluorescence) and Snail (red fluorescence) levels, compared to the NC treatment in all EA PCa cells. In AA PCa (MDA PCa 2b) cells, transfection of miR-99b-5p caused the inhibition of Snail protein level, but did not significantly change Vimentin expression, as compared to its NC treatment (FIG. 20D, middle panels). Similarly, Enz or Abi treatment resulted in a reduction in Snail in all EA and AA PCa cell lines, as compared to the NC treatment (FIGS. 20A-20D, Snail panels). Compared to NC treatment, Enz or Abi treatment caused a reduction in Vimentin protein levels in all EA PCa cells but not AA PCa cells (FIGS. 20A-20D, Vimentin panels). Notably, miR-99b-5p/Enz or miR-99b-5p/Abi treatment further downregulated Snail and Vimentin in all three EA PCas, compared to either miR-99b-5p or Enz/Abi as single agents (FIGS. 20A-20C). In AA PCa, Snail (but not Vimentin) expression levels were significantly inhibited in miR-99b-5p/Enz and miR-99b-5p/Abi combination vs. single agents of miR-99b-5p, Enz, or Abi (FIG. 20D).

Immunoblotting Validation of N-Cadherin, E-Cadherin, Vimentin, and Snail Protein Levels of EA and AA PCa Cells in Response to miR-99b-5p Mimic, Enz, Abi, miR-99b-5p Enz, and miR-99b-5p Abi

To verify the immunofluorescence assay results (FIGS. 19A-19D and FIGS. 20A-20D), Western blot analyses of the EMT markers were performed using cell lysates from EA PCa (LNCaP, 22Rv1, and C4-2B) and AA PCa (MDA PCa 2b) cells treated with NC, miR-99b-5p mimic, Enz, Abi, miR-99b-5p/Enz, and miR-99b-5p/Abi, respectively.

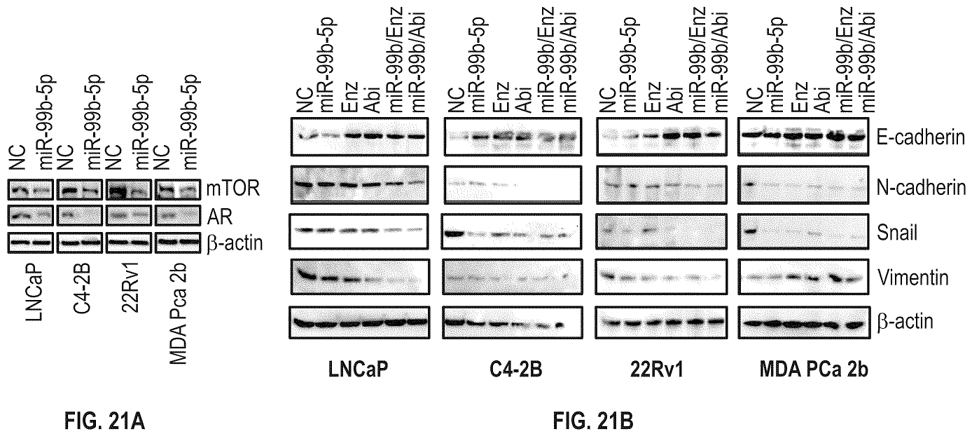

First, the cell lysates from all cell lines transfected with NC and miR-99b-5p mimic were subjected to Western blot for examining mTOR and AR protein levels. As shown in FIG. 21A, overexpression of miR-99b-5p resulted in downregulation of mTOR and AR in all EA and AA PCa cell lines, confirming that MTOR and AR were targeted and inhibited by miR-99b-5p, as previously described. Second, the cell lysates from EA and AA PCa under all six treatments were collected and subjected to examine the protein expression levels of E-cadherin (epithelial), Snail (EMT-TF), N-cadherin, and Vimentin (mesenchymal). As shown in FIG. 21B, E-cadherin was upregulated, while Snail and N-cadherin were downregulated upon treatments of miR-99b-5p mimic, Enz, or Abi. Similar to the immunofluorescence staining results, an additive inhibitory effect of Snail and N-cadherin and additive upregulated effect of E-cadherin was observed in all EA and AA PCa cells under combined treatment (miR-99b-5p/Enz or miR-99b/Abi) vs. single agent (miR-99b-5p, Enz, or Abi alone). Vimentin (mesenchymal marker) was also downregulated upon miR-99b-5p mimic, Enz, Abi, miR-99b-5p/Enz, or miR-99b-5p/Abi in EA PCa cell lines (but not in AA PCa cell line, MDA PCa 2b) (FIG. 21B).

These results are consistent with the immunofluorescence assay results, suggesting that miR-99b-5p, Enz, and Abi may function as single agents and synergize in combination to inhibit activation of EMT in EA and AA PCa cells.

Wound Healing Assays Revealed that miR-99b-5p Mimic and Enz Abi Treatment Suppress the Migration of EA and AA PCa Cell Models

To further test whether inhibition of EMT by miR-99b-5p/Enz/Abi affects the cell migration of EA and AA PCa, we performed scratch assays to examine migration capacities/mobilities of EA and AA PCa cells treated with miR-99b-5p, Enz, Abi, miR-99b-5p/Enz, and miR-99b-5p/Abi. Specifically, the migration rates of the EA and AA PCa (under different treatments) were determined in a window of 48 h.

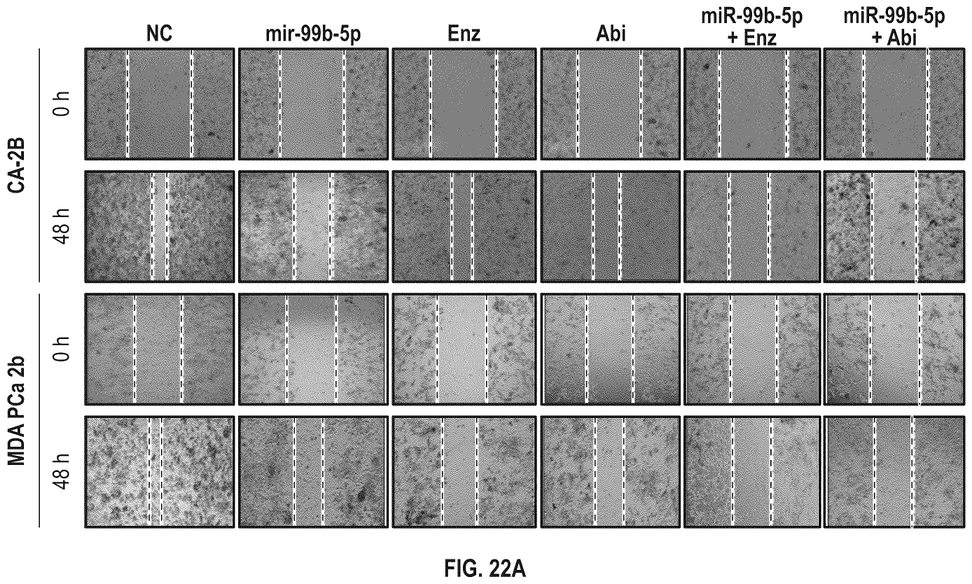

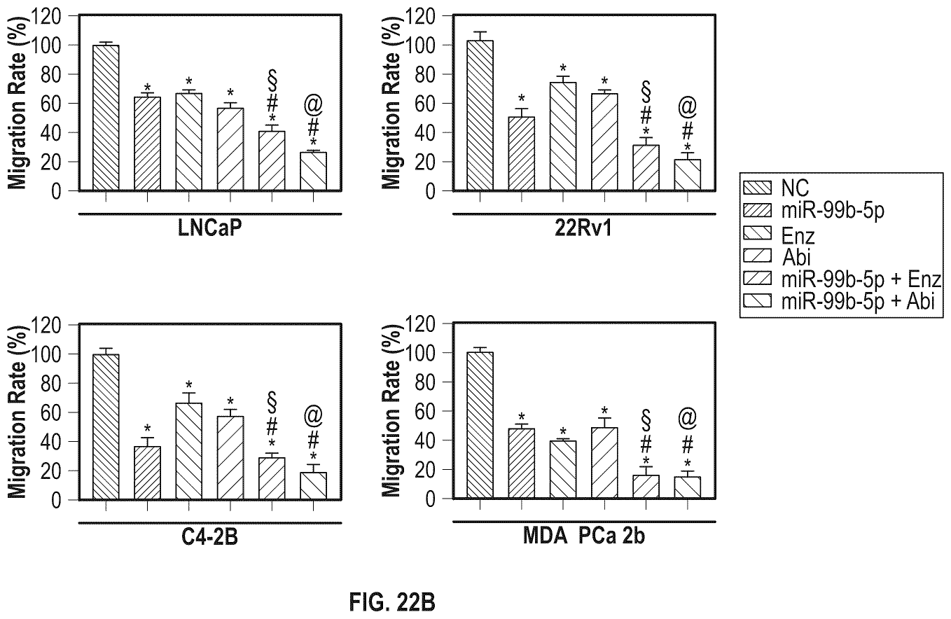

Compared to the NC-treated cells, miR-99b-5p-, Enz-, or Abi-treated cells resulted in a generalized lower migration capacity (with wider spaces between the migrating tumor cells at 48 h, defined by the two yellow dashed lines) of EA and AA PCa cells. Additionally, the treatment combination (miR-99b-5p/Enz or miR-99b-5p/Abi) synergizes the inhibitory effects on tumor migrations in all EA and AA PCa cell lines (FIG. 22A). By calculating the migration distance per 48 h (defined as migration rate in this study), bar graphs showing the migration rates of the PCa cells in all treatments are presented in FIG. 22B. Compared to the NC treated cells (migration rate was defined as 100%), mi-99b-5p treatment caused a generalized reduction (with 40-60% decrease) in migration rates. Enz and Abi treatments resulted in a 35-55% reduction in migration rates in LNCaP (androgen-sensitive EA PCa) and MDA PCa 2b (AA PCa) and a slighter suppression (with 25-30% decrease) of migration rates in EA CRPC (22Rv1 and C4-2B) cells. Notably, the combination of miR-99b-5p mimic with either Enz or Abi synergistically inhibits migration rates (with a 60-85% decrease in migration rates) in all the EA and AA PCa cells. These results strongly suggested that miR-99b-5p, Enz, and Abi can individually and synergistically inhibit tumor migration as single agents and in combinations (likely through the suppression of EMT activation).

Overexpression of miR-99b-5p and Enz/Abi Negatively Regulates EMT-Mediated Migration in PCa Cell Lines Based on Transwell Assays

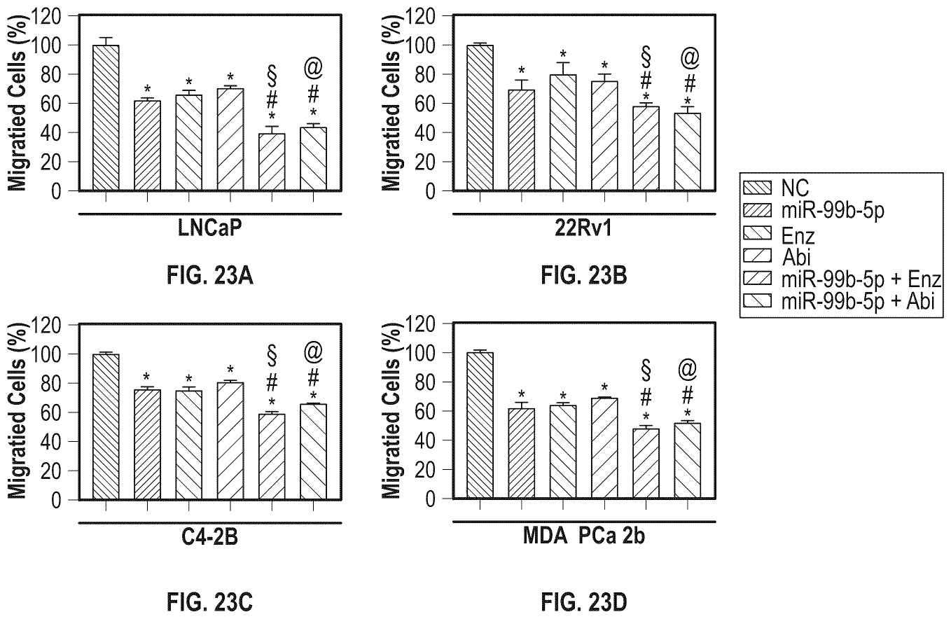

It is evident that tumor dissemination initiates with tumor invasion through the basement membranes, followed by migration to surrounding tissues, intravasation into blood vessels, and finally achieving tumor migration and colonization at distant organ sites. The transwell assay, an in vitro assay mimicking the migration process, was utilized to evaluate the tumor migration capacities of PCa cells under miR-99b-5p and/or Enz/Abi treatments. Specifically, the efficacies of miR-99b-5p, Enz, and Abi (as single agents or in combinations) on inhibiting the migration capacities of LNCaP, 22Rv1, C4-2B, and MDA PCa 2b cells were assessed using transwell assays (FIGS. 23A-23D). As shown in FIGS. 23A-23D, miR-99b-5p mimic, Enz, and Abi as single agents resulted in a generalized reduction (25-40%, 20-35%, and 20-30% decrease, respectively) in migration capacities when compared to NC treatments in EA and AA PCa cells. On the other hand, combination of miR-99b-5p with Enz further suppressed tumor migration capacities when compared to miR-99b mimic (with an additional 15-25% decrease) or Enz alone (with an additional 20-30% decrease) in EA and AA PCa cells. Similarly, a combination of miR-99b-5p mimic with Abi further suppressed tumor migration by an additional 10-20% and 10-30% decrease in migration capacities, when compared to miR-99b-5p mimic or Abi alone, respectively, in EA and Aa PCa cells (FIGS. 23A-23D). Taken together, the cell migration assay results have suggested that miR-99b-5p mimic, Enz, and Abi (as single agents or in combination) suppress EMT-mediated cell migration of EA and AA PCa cells.

Treatment of miR-99b-5p Mimic or Enz Abi Reduces Cancer Cell Adhesion of PCa Cells

Cell adhesion to the extracellular matrix (ECM) is one of the critical steps promoting tumor growth, invasion, and metastasis. To test how miR-99b-5p and Enz/Abi treat-ments affect tumor cell adhesion to ECM, a series of cell adhesion assays were conducted in EA and AA PCa cells treated with NC, miR-99b-5p, Enz, Abi, miR-99b-5p/Enz, and miR-99b-5p/Abi. Specifically, PCa cells transfected with nonsense RNA or miR-99b-5p mimic were grown on the ECM-coated plate, and were then treated with vehicle, Enz, or Abi. The assays allowed us to visualize the efficiencies of PCa cell adherence on ECM in response to different treatments.

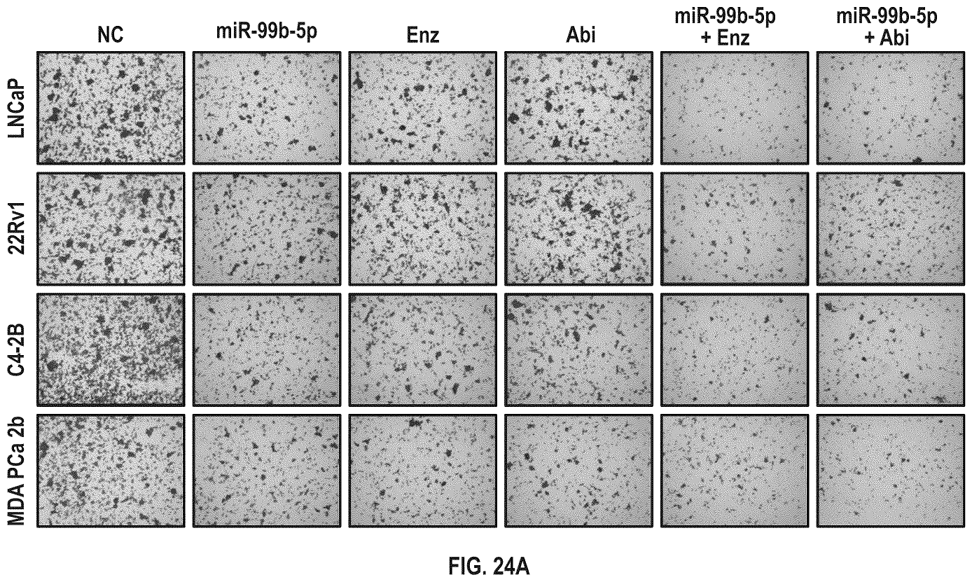

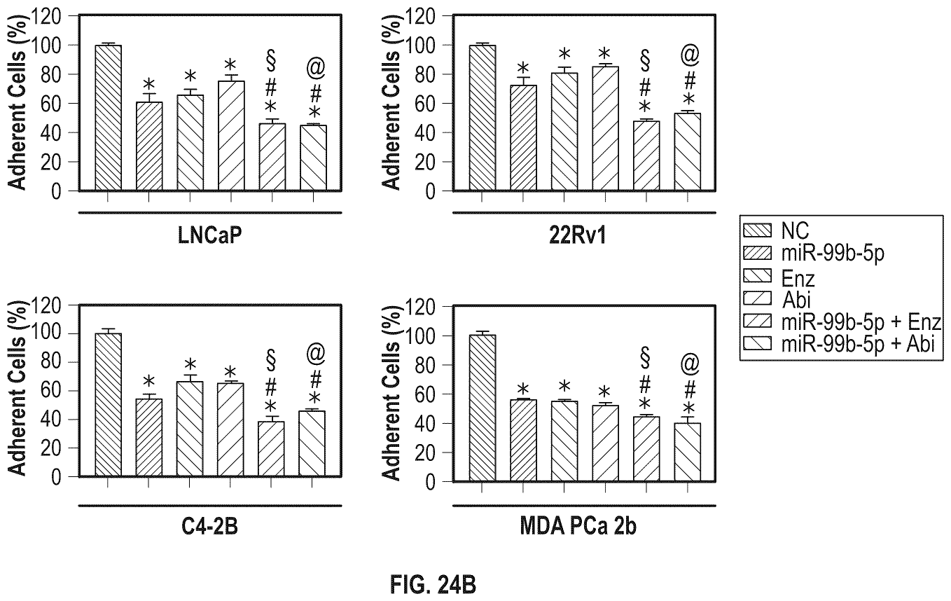

As shown in FIG. 24A, we observed that transfection of miR-99b-5p resulted in a reduction in cell adhesion capacities (with a 25-45% decrease, FIG. 24B) in EA and AA PCa cells, as compared to NC. On the other hand, Enz and Abi treatments resulted in a generalized reduction (with a 20-50% decrease) in cell adhesion capacities of the EA and AA PCa cells, when compared to the NC groups. Similar to the migration and cell adhesion assays, the combined treatment of miR-99b-5p/Enz or miR-9b-5p/Abi induced a synergistic inhibition (with a 50-65% reduction) of cell adhesion capacities in the EA and AA PCa cell models, when compared to either miR-99b-5p, Enz, or Abi alone. Taken together, these results suggest that treatment of miR-99b-5p, Enz, and Abi as single agents or in combinations may individually or synergistically disrupt PCa cell adhesion to ECM.

Treatment of miR-99b-5p Mimic and Enz/Abi Modulates Angiogenesis Process in PCa Cells

HUVEC is an endothelial cell line with an ability to form capillary-like projections under in vitro conditions. It has been shown that culturing HUVEC cells with ECM components facilitates the development of a cellular branched structure and tubule-like projections, mimicking the in vivo process of angiogenesis.

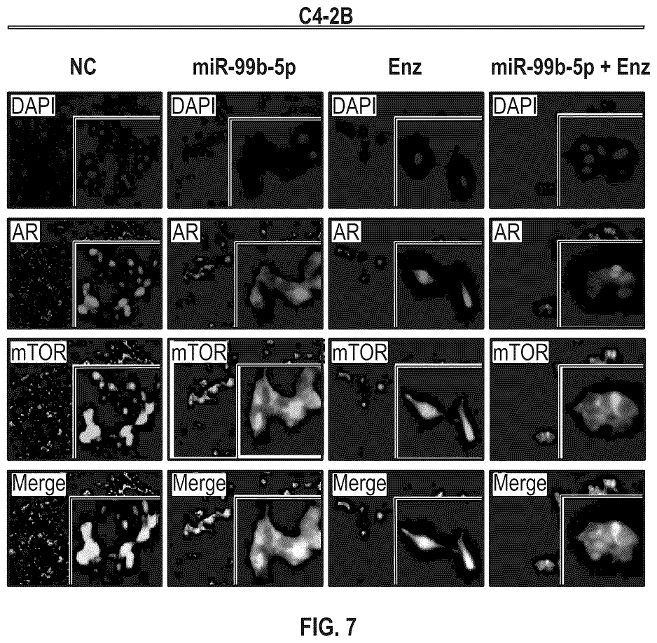

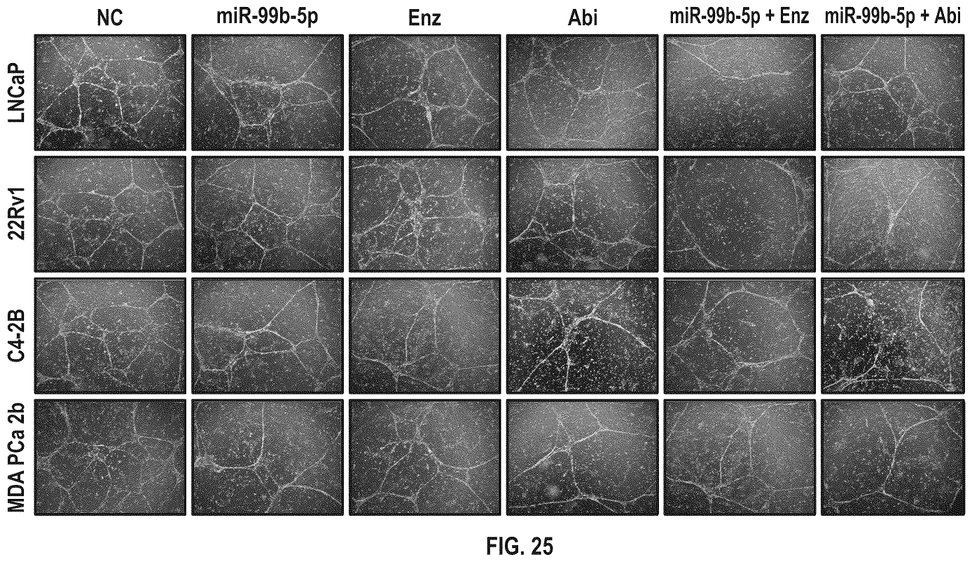

In this study, tubule formation efficiency was employed as an index to evaluate the angiogenesis capacities of the PCa cells under different treatments. First, HUVEC cells were exposed to the condition medium (CM) derived from PCa cells treated with NC, miR-99b-5p, Enz, and Abi and then they were subjected to tube formation assays. As shown in FIG. 7, HUVEC cells successfully developed tubule-like structures after being incubated with CM derived from NC-treated PCa cells. In contrast, miR-99b-5p mimic, Enz, and Abi as single agents caused a slight to moderate reduction in tube formation in all PCa cells when compared to the NC groups. Moreover, miR-99b-5p/Enz and miR-99b-5p/Abi combinations resulted in a more significant reduction in tube formation, as compared to the single agent treatment with miR-99b-5p mimic, Enz, or Abi alone (FIG. 25). These results have implicated that miR-99b-5p, Enz, and Abi may inhibit angiogenesis, and miR-99b-5p/Enz and miR-99b-5p/Abi combinations further synergize the inhibition of the angiogenesis process in PCa.

Discussion for a Combination of miR-99b-5p and Enzalutamide or Abiraterone Synergizes the Suppression of EMT-Mediated Metastasis in Prostate Cancer

EMT is a molecular cellular program that is required for cell development, wound healing, fibrosis, and cancer progression/metastasis. Previous studies have also highlighted the critical role of EMT plasticity in PCa metastasis and treatment resistance. Several signaling pathways have been identified as upstream regulators for activation of EMT in cancers, including Wnt, Notch, TGFβ, PI3K/AKT/mTOR, JAK/STAT, and hypoxia/HIF-la signaling pathways. These cell-intrinsic signaling pathways cooperate to induce the transcriptional activation of EMT-TFs, such as SNAIL, ZEB1/2, and Twist1/2, subsequently triggering the EMT process for induction of the transition to the mesenchymal state of the tumor cells.

Numerous studies have revealed that miRNAs function in promoting or inhibiting PCa metastasis. For instance, miR-9, mR-21, and miR-181a have been shown to promote EMT, while miR-34, miR-130b, miR-200b, miR-204, and miR-573 have been involved in suppressing EMT. Our previous studies have highlighted the deregulated miRNA-mRNA interaction as one of the critical epigenomic factors regulating PCa aggressiveness and treatment resistance. Among the identified reciprocal miRNA/mRNA pairings involved in aggressive PCa, miR-99b-5p/MTOR (upregulated/downregulated) pairing has been revealed as a central miRNA/mRNA pairing coordinating PI3K/AKT/mTOR signaling with HIF-la and VEGF pathways. TGFβ collaborates with PI3K/AKT signaling to activate mTOR and NFκB, activating the expression of EMT-TF genes. A previous study has also shown that mTORC1 and mTORC2 promote EMT-mediated metastasis through activation of RhoA and Rac1 in colorectal cancer.

In this study, restoring miR-99b-5p (which is downregulated in AA PCa and CRPC) resulted in downregulation of mTOR, theoretically inhibiting the mTOR/NFκB-mediated expression of SNAIL1/2, ZEB1/2, and TWIST1/2. This hypothesis was validated by our immunofluorescence staining and Western blot assays of Snail protein levels in NC vs. miR-99b-5p mimic-treated PCa cells (FIGS. 20A-20D and FIGS. 21A-21B). Snail is an EMT-TF that negatively regulates the expression of E-cadherin. Theoretically, overexpression of miR-99b-5p (in this study) would cause inhibition of mTOR-mediated activation of Snail, thereby leading to reactivation of E-cadherin, reversing the mesenchymal state to the epithelial state, and inhibiting metastasis of PCa. The upregulation of E-cadherin (epithelial marker); down-regulation of Snail (EMT-TF, and downstream gene of mTOR), Vimentin, and N-cadherin (mesenchymal markers); and reduction of migration capacity successfully validates the suppressive role of miR-99b-5p in regulating EMT and EMT-mediated metastasis in PCa. Downregulation of miR-99b-5p and upregulation of mTOR has also been shown to activate HIF-la and VEGF signaling in aggressive PCa. Although the role of hypoxia/HIF-la signaling in EMT remains unclear in PCa, HIF-la signaling has been implicated to induce EMT in other cancers. VEGF is one of components secreted by cancer-associated fibroblasts (CAFs) that promotes EMT in cancer. In addition, HIF-la-mediated activation of VEGF-A has been found to promote the induction of EMT. Thus, downregulation of miR-99b-5p is likely to activate HIF-la and VEGF signaling (via mTOR) and upregulation of VEGF-A (a miR-99b-5p target), subsequently leading to the induction of EMT in AA PCa and EA CRPC. Together, it explains why miR-99b-5p expression results in downregulation of EMT markers (FIGS. 19A-19D, FIGS. 20A-20D, and FIGS. 21A-21B), inhibition of metastasis (FIGS. 22A-22B, FIGS. 23A-23D, and FIGS. 24A-24B), and angiogenesis (FIG. 25) in PCa cells.

Androgen signaling is another pathway implicated in the regulation of EMT in PCa. Activation of the androgen receptor (AR) has been shown to inhibit E-cadherin expres-sion and promote activation of EMT, and androgen-mediated β-catenin signaling contributes to the induction of EMT in PCa. However, several studies have also implicated that ADT itself may contribute to the development of EMT, leading to PCa invasion/metastasis and CRPC progression/metastasis. In this study, we have demonstrated that miR-99b-5p expression can simultaneously target and inhibit the ex-pression of AR and MTOR, leading to the suppression of EMT-mediated metastasis in AA PCa and EA CRPC. Recently, Zheng et al. showed that androgen stimulation induces EMT-mediated metastasis in AR-positive PCa, but not AR-negative PCa cells. SiRNA knock-down of eIF5A leads to upregulation of E-cadherin and downregulation of N-cadherin and Vimentin in AR-positive PCa (such as VCap and 22Rv1). In addition, a genomic study by Fletcher et al. has revealed that inhibition of miR-346, miR-361-3p, and miR-197 resulted in inhibition of the AR expression level, leading to suppression of the EMT event and EMT-mediated metastasis in AR-positive LNCaP and C4-2 cells. Similar to these two studies, our study used four AR-positive PCa cell lines, LNCaP (metastatic EA PCa), C4-2B, and 22Rv1 (EA CRPC), and MDA PCa 2b (AA PCa), as our in vitro cell model to test the efficacies of miR-99b-5p alone and in combination with Enz or Abi in PCa. MiR-99b-5p mimic directly targets/inhibits the expression of AR, instead of modulating AR activity via AR antagonist, which may avoid inversely inducing/promoting EMT (i.e., in AR-negative PCa) and more effectively inhibit AR signaling and AR-mediated EMT in PCa, especially in AR-positive CRPC.

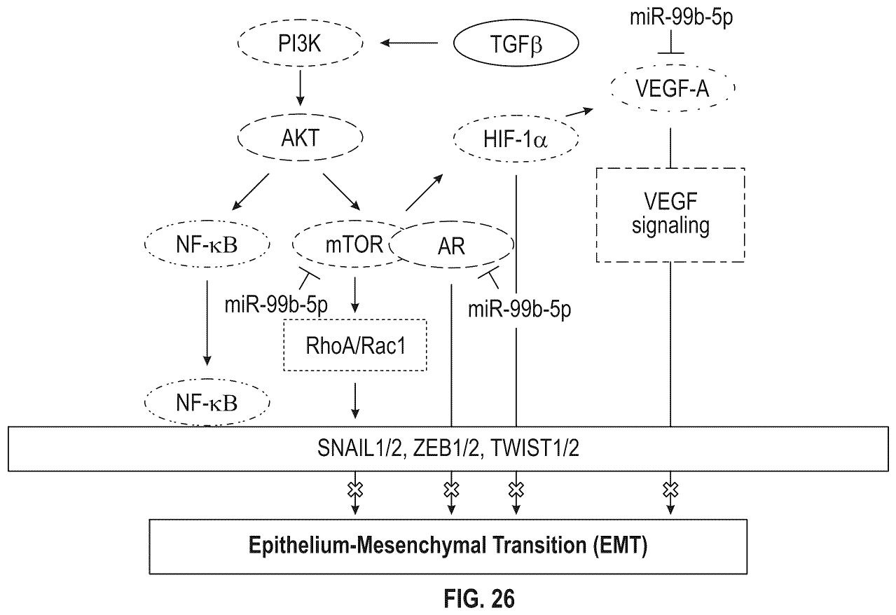

An illustration representing the miR-99b-5p-mediated inhibition of EMT is presented in FIG. 26. To date, this is the first study to demonstrate the involvement of miR-99b-5p in the regulation of EMT, through modulating AR, mTOR, HIF1α, and VEGF signaling.

### CONCLUSIONS

In conclusion, the present disclosure demonstrates miR-99b-5p as a potent EMT-suppressive miRNA that inhibits EMT-mediated metastasis/angiogenesis, through simultaneously targeting AR/MTOR and downregulating mTOR, AR, HIF-1, and VEGF signaling in AR-positive PCa. Further elucidating the molecular mechanism of the miR-99b/mTOR/AR signaling axis in regulating EMT-mediated metastasis will warrant the development of novel therapeutic strategies for aggressive PCa, such as AA PCa and CRPC. Despite the promising potential for miR-99b-5p mimic in the inhibition of EMT in AA PCa and CRPC, challenges remain in clinical application. Further developing an efficient delivery system, generating a chemically stable miR-99b-5p mimic, and minimizing the off-target effect of miR-99b-5p will facilitate the development of this miRNA-based therapy for treating CRPC in preclinical and clinical settings.

From the foregoing, it can be seen that the present disclosure accomplishes at least all of the stated objectives.

### Glossary

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

The mammalian target of rapamycin (“mTOR”), also referred to as the mechanistic target of rapamycin, and sometimes called FK506-binding protein 12-rapamycin-associated protein 1 (FRAP1), is a kinase that in humans is encoded by the MTOR gene. mTOR is a member of the phosphatidylinositol 3-kinase-related kinase family of protein kinases. mTOR links with other proteins and serves as a core component of two distinct protein complexes, mTOR complex 1 and mTOR complex 2, which regulate different cellular processes. In particular, as a core component of both complexes, mTOR functions as a serine/threonine protein kinase that regulates cell growth, cell proliferation, cell motility, cell survival, protein synthesis, autophagy, and transcription. As a core component of mTORC2, mTOR also functions as a tyrosine protein kinase that promotes the activation of insulin receptors and insulin-like growth factor 1 receptors. mTORC2 has also been implicated in the control and maintenance of the actin cytoskeleton.

The androgen receptor (“AR”), also known as NR3C4 (nuclear receptor subfamily 3, group C, member 4), is a type of nuclear receptor that is activated by binding any of the androgenic hormones, including testosterone and dihydrotestosterone, in the cytoplasm and then translocating into the nucleus. The androgen receptor is most closely related to the progesterone receptor, and progestins in higher dosages can block the androgen receptor. The main function of the androgen receptor is as a DNA-binding transcription factor that regulates gene expression; however, the androgen receptor has other functions as well. Androgen-regulated genes can be critical for the development and maintenance of the male sexual phenotype.

“SMARDCD1” is a subunit of the SWI/SNF complex. SWI/SNF-related matrix-associated actin-dependent regulator of chromatin subfamily D member 1 is a protein that in humans is encoded by the SMARCD1 gene. The protein encoded by this gene is a member of the SWI/SNF family of proteins, whose members display helicase and ATPase activities and which are thought to regulate transcription of certain genes by altering the chromatin structure around those genes.

What is claimed is:

1. A therapeutic combination for inhibiting cancer metastasis and angiogenesis comprising:
miR-99b-5p; and
an androgen receptor antagonist that simultaneously targets an androgen receptor (AR) and mammalian target of rapamycin (mTOR) signaling.

2. The therapeutic combination of claim 1, wherein the androgen receptor antagonist comprises enzalutamide (Enz) or abiraterone (Abi).

3. The therapeutic combination of claim 1, wherein the miR-99b-5p and the androgen receptor antagonist:
inhibit epithelial-mesenchymal transition (EMT) in prostate cancer (PCa);
are configured to upregulate E-cadherin;
are configured to downregulate Snail;
are configured to downregulate N-cadherin; or
are configured to downregulate Vimentin.

4. The therapeutic combination of claim 1, wherein the miR-99b-5p regulates a cellular process selected from a group consisting of: cell growth, cell proliferation, cell motility, cell survival, protein synthesis, and autophagy.

5. The therapeutic combination of claim 4, wherein the cellular process is transcription and the miR-99b-5p regulates the transcription of a gene by altering a chromatin structure of the gene.

6. A tumor suppressive regulator comprising:
miR-99b-5p, wherein the miR-99b-5p functions as an epigenomic regulator.

7. The tumor suppressive regulator of claim 6, further comprising enzalutamide (Enz) to enhance cytotoxicity of the tumor suppressive regulator against cancer.

8. The tumor suppressive regulator of claim 6, wherein the miR-99b-5p regulates a cellular process selected from a group consisting of: cell growth, cell proliferation, cell motility, cell survival, protein synthesis, and autophagy.

9. The tumor suppressive regulator of claim 8, wherein the cellular process is transcription and the miR-99b-5p regulates the transcription of a gene by altering a chromatin structure of the gene.

10. The tumor suppressive regulator of claim 6, wherein the miR-99b-5p regulates a nuclear receptor by binding a hormone thereto.

11. The tumor suppressive regulator of claim 10, wherein the nuclear receptor is the androgen receptor (AR) and the hormone is selected from the group consisting of testosterone and dihydrotestosterone.

12. The tumor suppressive regulator of claim 6, wherein the miR-99b-5p modulates the mTOR/AR/SMARCD1 signaling axis.

13. A method of diagnosing cancer, the method comprising:
using miR-99b-5p as a biomarker to identify presence of prostate cancer.

14. The method of diagnosing cancer of claim 13, further comprising inhibiting cell proliferation/survival using the miR-99b-5p.

15. The method of diagnosing cancer of claim 13, further comprising inducing cell apoptosis in cancer cells.

16. The method of diagnosing cancer of claim 13, further comprising determining the cancer is a castration resistant cancer.

17. The method of diagnosing cancer of claim 13, further comprising treating the cancer with a hormone-based treatment.

18. The method of diagnosing cancer of claim 13, further comprising further comprising enhancing cytotoxicity against the cancer with enzalutamide (Enz).

19. A method of treating the prostate cancer diagnosed by way of the method of claim 8, comprising:
inhibiting cell proliferation/survival using miR-99b-5p; and
inducing cell apoptosis in cancer cells.

20. The method of claim 14, wherein the method is practiced in vitro.
